SEQUENCE LISTING (12 sequences)

SEQ ID NO: 1 - DNA, 47 bp, Artificial Sequence
  Exemplary Adapter for RNA sequence
  misc_feature (21)..(25): n is a, c, g, or t
  misc_feature (47)..(47): n is a, c, g, or t
gtctcgtgggctcggatcgtnnnnnttttttttttttttttttttvn

SEQ ID NO: 2 - DNA, 15 bp, Artificial Sequence
  Exemplary Amplification primer
gtctcgtgggctcgg

SEQ ID NO: 3 - DNA, 22 bp, Artificial Sequence
  Annealing sequence
  misc_feature (22)..(22): n is a, c, g, or t
ttttttttttttttttttttvn

SEQ ID NO: 4 - DNA, 44 bp, Artificial Sequence
  One-Tn5 Exemplary Adapter for DNA sequence
  misc_feature (21)..(25): n is a, c, g, or t
gtctcgtgggctcggtcatgnnnnnagatgtgtataagagacag

SEQ ID NO: 5 - DNA, 30 bp, Artificial Sequence
  Exemplary Adapter for RNA sequence
  misc_feature (21)..(25): n is a, c, g, or t
  misc_feature (30)..(30): n is a, c, g, or t
gtctcgtgggctcggatcgtnnnnnggghn

SEQ ID NO: 6 - DNA, 30 bp, Artificial Sequence
  Exemplary Adapter for RNA sequence
  misc_feature (21)..(25): n is a, c, g, or t
  misc_feature (30)..(30): n is a, c, g, or t
gtctcgtgggctcggatcgtnnnnntttvn

SEQ ID NO: 7 - DNA, 26 bp, Artificial Sequence
  Exemplary amplification primer
gatgtgtggaggtctcgtgggctcgg

SEQ ID NO: 8 - DNA, 19 bp, Artificial Sequence
  Mosaic Sequence
  modified_base (1)..(1): Phosphorylated
ctgtctcttatacacatct

SEQ ID NO: 9 - DNA, 33 bp, Artificial Sequence
  Read1-Tn5 sequence/Read 1 primer
tcgtcggcagcgtcagatgtgtataagagacag

SEQ ID NO: 10 - DNA, 34 bp, Artificial Sequence
  I7 index primer
ccgagcccacgagacctgtctcttatacacatct

SEQ ID NO: 11 - DNA, 33 bp, Artificial Sequence
  I5 index primer
ctgtctcttatacacatctgacgctgccgacga

SEQ ID NO: 12 - DNA, 34 bp, Artificial Sequence
  Read 2 primer
agatgtgtataagagacaggtctcgtgggctcgg

(19) United States
(12) Patent Application Publication
(10) Pub. No.: US 2023/0366009 A1
(43) Pub. Date: Nov. 16, 2023

(54) SIMULTANEOUS AMPLIFICATION OF DNA AND RNA FROM SINGLE CELLS

(71) Applicant: THE HONG KONG UNIVERSITY OF SCIENCE AND TECHNOLOGY, Hong Kong (CN)

(72) Inventors: Angela Ruohao WU, Hong Kong (CN); Lei YU, Hong Kong (CN)

(21) Appl. No.: 18/246,529

(22) PCT Filed: Oct. 19, 2021
(86) PCT No.: PCT/IB2021/000713

§ 371 Date: Mar. 24, 2023

Related U.S. Application Data
(60) Provisional application No. 63/093,368, filed on Oct. 19, 2020.

(51) Int. Cl. C12Q 1/6806; C12N 15/10; C12Q 1/48

(52) U.S. Cl. CPC C12Q 1/6806; C12N 15/1096; C12Q 1/485

(57) ABSTRACT

Provided is a method for simultaneous identification of multiple target biomolecule types from a single cell or nucleus. Methods are conducted using molecular biology-based tagging and amplification of DNA and RNA from the same cell, in a single-container reaction, generating a library of amplified nucleic acids that can be sequenced. Any nucleic acids in the form of DNA or RNA in the cell can be tagged and co-amplified using the same method, including DNA or RNA tags attached to other biomolecules.

### CROSS-REFERENCE TO RELATED APPLICATION

This application claims the benefit of U.S. Provisional Application Ser. No. 63/093,368, filed Oct. 19, 2020, which is hereby incorporated by reference in its entirety including any tables, figures, or drawings.

### BACKGROUND OF THE INVENTION

Single-cell genomics has become a mainstay technology used to dissect multicellular organisms and tissues that are composed of cells with diverse functions¹⁻⁴. The power of this approach has been demonstrated in several cell atlas studies: novel cell types have been discovered that further led to the elucidation of new mechanisms; complex cellular interactions and transitions associated with disease initiation or progression have been revealed; cross-species analyses have shed light on evolutionary processes⁵⁻⁷. The use of single-cell technology in studying cancer is especially important. Regulatory mechanisms underlying drug resistance or immune evasion are elusive and complex, and tumor cell heterogeneity tumor is a major contributing factor to this complexity, making it particularly challenging to dissect these mechanisms with bulk techniques⁸⁻¹⁰. Single-cell technologies have greatly enhanced our understanding of tumor heterogeneity and accelerated mechanistic discovery. At the phenotype level, single-cell RNA-seq (scRNA-seq) has been used to uncover drug-resistant melanoma subpopulations and to characterize cancer stem cell subpopulations in glioblastoma^{7,11-14}. scRNA-seq has also enabled a more comprehensive phenotypic understanding of the tumor microenvironment (TME) in many cancers including glioma and colorectal cancer¹⁵⁻¹⁹. At the genotype level, genomic instability contributes to cancer initiation, progression, relapse, and metastasis⁸. With single-cell whole genome sequencing (scWGS), the clonal structure of the tumor can be resolved, and evolutionary analysis based on copy number aberrations (CNAs) can reveal tumor progression^{20,21}.

Evidently, both the genomic and transcriptomic heterogeneity of tumors contributes to the disease, and understanding the importance of both in cancer studies is crucial. Several single-cell methods that interrogate DNA and RNA simultaneously in the same cell (scWGS-RNAseq) have been developed²²⁻²⁶. However, these first-generation scWGS-RNAseq methods have not been widely applied since their invention, largely because they require physical separation of DNA and RNA, often by physical separation of the nucleus from the cytoplasm, sometimes by physical separation of polyadenylated RNA from the rest of the cell by polyT-bead based fishing. These separation techniques are labor-intensive and technically demanding, time-consuming, requiring well-trained experimental techniques, or require special microfluidic devices. Furthermore, they are not applicable to frozen samples, where it is impossible to obtain intact single-cell suspensions. As such, existing scWGS-RNAseq methods cannot be applied to the vast majority of primary biobanked tumor samples. All in all, these limitations make first generation scWGS-RNAseq methods not easily accessible.

Therefore, there remains a need for methods of co-profiling of DNA-encoded and RNA-encoded information from a single cell.

### BRIEF SUMMARY OF THE INVENTION

The present invention relates to a novel single-cell DNA and RNA co-amplification method, scONE-seq, which enables co-profiling of the transcriptome and genome from the same single cell or nucleus in a one-container reaction. In certain embodiments, the invention pertains to a barcoding strategy that introduces a 6-base long DNA-specific and RNA-specific barcodes to each type of nuclei acid during the single-cell DNA/RNA amplification process, while also incorporating unique molecular identifiers (UMIs)²⁷⁻²⁹. Thus, DNA and RNA reads can be amplified together by a shared primer region, but later distinguished in-silico by their respective specific barcode information after sequencing. Compared to the first generation scWGS-RNAseq methods, scONE-seq has several advantages: it has a simplified library construction workflow; it is compatible with standard biology workflows such as fluorescence-activated cell sorting (FACS); being a one-pot (i.e., one container or one tube) reaction, its throughput can be easily scaled up using liquid-handling robots; most importantly, scONE-seq does not require physically separating the DNA and RNA, and is therefore applicable to a variety of sample types including single nuclei. In certain embodiments, frozen clinical samples and cell types that are difficult to dissociate into single-cell suspensions, which are intractable with scDR-seq methods, can be profiled using scONE-seq.

The method is a DNA/RNA barcoding strategy, which tags the DNA and RNA with different nucleic acid barcodes respectively prior to single cell DNA/RNA co-amplification. Amplification adapters can also be added and used to co-amplify the DNA and reverse transcribed cDNA with the same primer sets, generating the sequencing library. After sequencing, DNA and RNA reads can be computationally distinguished by demultiplexing of the barcode.

The methods of the invention can be useful for any type of cell. Methods of the invention can be applied to the co-profiling of whole genomes and total transcriptomes from a single cell. The methods are particularly useful for studying diseases such as cancer, in which the genome and transcriptome reflect different facets of the disease progression. The methods can also be used to study viral activity within cells, as infected cells harbor viral DNA and RNA in addition to the endogenous genome and transcriptome. The subject methods can further be used identify bacteria and their interactions with phage. In certain embodiments, methods can also be used to screen for drugs and discover new drugs or drug functions. In certain embodiments, the methods can be used with microfluidics devices.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1K. Overview of scONE-seq: schematic and benchmark. FIG. 1A, Workflow of scONE-seq. DNA and RNA barcodes are added by Tn5 with custom adaptor and RT primers respectively. Read2-Illumina sequencing primer is included in this pre-amplification process. Read1-Illumina sequencing primer is added in the later library construction step with a second Tn5 tagmentation custom adaptor. FIG. 1B, Gene detection sensitivity for scONE-seq (n=86, HCT116 cells) and Smart-seq2 (n=94, HCT116 cells), downsampled to similar sequencing depth (0.2 million mapped reads) (P<2×10⁻¹⁶, t-test). Shown are numbers of genes detected over 1 count. FIG. 1C, Gene body coverage for scONE-seq (n=86) and Smart-seq2 (n=94). Cell-cell variations are shown with error areas. FIGS. 1D-1G, Accuracy across mock samples for scONE-seq (RNA control, DNA+RNA group) and Smart-seq2. Pearson correlations were calculated from log-transformed TPM values. FIG. 111, The Lorenz curve of bulk and scONE-seq single-cell data. Percentiles of the genome covered are plot against the cumulative fraction of reads. A perfect coverage uniformity results in a straight line with slope 1. FIG. 1I-K, Dots plot with normalized counts across the genome and solid line plots for corresponding estimated integer copy number. Amplification regions are highlighted with red; deletion regions are highlighted with light blue. Data from the bulk HCT116 whole genome sequencing (FIG. 1I), the HCT116 scONE-seq pseudo bulk data (FIG. 1F=J, n=86), and a single-cell HCT116 scONE-seq data (FIG. 1K).

FIGS. 2A-2H. scONE-seq cell-types classification and CNAs clone identification. FIG. 2A—UMAP of scONE-seq cell lines RNA data, cells from same the cell line are clustered together; FIG. 2B—Differential expression genes (DEGs) heatmap; DEGs separate the cells based on their cell type; common markers for these cell lines are labeled in the heatmap. FIGS. 2C and 2E: UMAP of lymphocytes RNA data from scONE-seq (FIG. 2C) and Smart-seq2 (FIG. 2E); cell-types annotations are based on known markers of immune cells. Cell-type composition shows no difference between two datasets (P=0.5109, Chi-squared test). FIGS. 2D and 2F: Dot plots of markers used for cell-types annotations with the scONE-seq dataset (FIG. 2D) and Smart-seq2 dataset (FIG. 2F). FIG. 2G, Copy number profiles calculated with scONE-seq cell line DNA dataset; cells are organized by hierarchical clustering (normal n=27; HCT116 n=48; NPC43 n=108). FIG. 211, The minimum evolution tree with diploid as root; NPC43 cells used in this study acquired more CNAs compared with the genome state when the cell line was established. Unit shows the Manhattan distance.

FIGS. 3A-3G scONE-seq reveals the clonal composition of the IDH1-mutant astrocytoma. FIG. 3A: Schematic showing the patient history for the sample used in this study. The patient has been diagnosed as IDH1-mutant (Grade IV) astrocytoma. Surgery was performed to excise the tumor, and concurrent chemoradiotherapy (CCRT) was then applied. The recurrences of the tumor were excised without further drug treatment. Tumor specimens were snap-frozen in the liquid nitrogen tank and stored for two years before subject to nuclei extraction.; FIG. 3B: UMAP of scONE-seq DNA copy number data shows the 4 genome states in this IDH1-mutant astrocytoma sample. 2R—second recurrence. FIG. 3C: Heatmap of integer copy numbers of all the 2R astrocytoma cells profiled; 3 clones with different copy number profiles are observed. The bottom annotation bar represents the CNAs in this tumor sample. Some commonly known glioma/astrocytoma driver genes are shown. Amplified genes are highlighted with red; deleted genes are highlighted with dark blue. FIG. 3D: The minimum evolution tree with diploid as root; the WES data inferred CNV from the same patient were integrated to show the evolutionary relationship between the tumor recurrences and their various clones. The P clones are inferred from bulk WES data (see Methods). P—primary; 1R—first recurrence. FIGS. 3E-3G: FIG. 3E shows the dots plot with normalized counts across the human genome and the solid line representing the estimated integer copy number. FIG. 3G shows the mirrored BAF dots plot across the genome. Relatively amplified regions are highlighted with red; relatively deleted regions are highlighted with blue. If dots are close to the red belt in the mirrored BAF dots plot, this indicates that there are LOH in those regions. If dots are close to the blue belt in the mirrored BAF dots plot, this indicates that there are imbalanced haplotypes in that regions. The top bar highlights the LOH regions of the genome. The clonal pseudo-bulk genome information for each 2R clone is also shown.

FIGS. 4A-4E. scONE-seq reveals the tumor microenvironment composition of an IDH1-mutant astrocytoma. FIG. 4A, UMAP of scONE-seq RNA data shows the TME composition cell types in this second recurrence IDH1-mutant astrocytoma sample. Tumor cells are classified into 4 cellular states based on their meta-module scores. FIG. 4B, Dot plot shows some markers for the annotation of cell types. FIG. 4C, UMAP of scONE-seq RNA data annotated with clonal information. The 2R clone 1 cells are clustered with normal astrocytes. FIG. 4D, Volcano plot shows the DEGs between the 2R clone 1 and clone 3 cells. Genes with higher expression in clone 1 cells are colored with red. Genes with higher expression in clone 3 are colored with blue. FIG. 4E, UMAP showing the integration of scONE-seq and 10× snRNA-seq dataset. The integrated data (left) retains all cell types identified with scONE-seq or 10× snRNA-seq. The split-UMAP (right) shows adequate mixing of cell-types found separately by the 2 methods, with clone 1 cells (from scONE-seq) and putative clone 1 cells (from snRNA-seq) falling into the same integrated cluster.

FIGS. 5A-5I. Characterization of 2R clone 1 features. FIG. 5A, The immunofluorescent images co-label the IDH1 (R132H) and ADCY8 in the FFPE section of the patient. The top panel shows images from the 2R tumor; the bottom shows images from the primary tumor from the same patient. The yellow arrow indicates the co-stained putative clone 1 cells; the red arrow indicates the other tumor cells; the Green arrow indicates normal astrocytes or GABAergic neurons. FIGS. 5B-5E, The AMPAR subunit-encoding gene expression pattern in this 2R IDH1-mutant astrocytoma. GRIA1 is highly expressed in clone 1 cells. FIGS. 5F-5I, The TGFβ signaling genes expression pattern in this 2R IDH1-mutant astrocytoma. TGFB2 is highly expressed in clone 1 cells and normal astrocytes. The receptors are mostly expressed in TAMs.

FIGS. 6A-6G. scONEseq benchmarking. FIG. 6A: Lorenz curve of single-cell scWGS data from DR-seq (SK-BR-3 cells), G&T-seq (mouse embryo 8-cells stage cells), and scONE-seq (frozen tumor nuclei) in a representative normal chromosome region (DR-seq and scONE-seq: human chr2; G&T-seq: mouse chr1 and chr19 chosen for comparable length to human chr2). Percentiles of the genome covered are plot against the cumulative fraction of reads. A perfect coverage uniformity results in a straight line with slope 1. Variations within each method are plotted using ±SD. scONE-seq is closest to the bulk control, indicating better uniformity for copy number calculation. FIG. 6B, Comparison of single-cell scWGS data dispersion from DR-seq (SK-BR-3 cells), G&T-seq (mouse embryo 8-cells stage cells), and scONE-seq (frozen tumor nuclei) in a representative normal chromosome region (DR-seq and scONE-seq: human chr2; G&T-seq: mouse chr1 and chr19 chosen for comparable length to human chr2). The box plot shows a significant technical improvement of scONE-seq over the other two methods, even though scONE-seq has lower average DNA sequencing depth in this comparison. FIG. 6C, Scatter plots show the correlation between detected quantity of ERCC spike-in and original input concentration of each ERCC molecule, comparing Smart-seq2 (1 μl 1:500,000 ERCC, n=3), scONE-seq (1 μl 1:500,000 ERCC, n=3), DR-seq (0.2 μl 1:500,000 ERCC, n=21), and G&T-seq (2 μl 1:500,000 ERCC, n=32). Of the methods compared, only scONE-seq shows comparable sensitivity and accuracy as Smart-seq2, an RNA-only amplification method. FIG. 6D, Gene detection sensitivity for mock scONE-seq RNA (n=3), mock scONE-seq DNA+RNA (n=3), and Smart-seq2 (n=3). Downsampled to similar sequencing depth (0.15 million mapped reads) (P=0.0019, ANOVA test). Shown are numbers of genes detected over 1 count. FIG. 6E, Precision evaluation with HCT116 cells. The most commonly expressed 8000 genes were used for scONE-seq (n=86) and Smart-seq2 (n=94) respectively. The box plots show the pairwise coefficient of determination (R2) values of cells with Pearson correlations from log-transformed TPM values. FIG. 6F, Estimated saturation plot with mock sample expression data from scONE-seq RNA, scONEseq DNA+RNA, and Smart-seq2. FIG. 6G, Estimated saturation plot with mock sample expression data from scONE-seq RNA, scONEseq DNA+RNA, and Smart-seq2.

FIGS. 7A-7AA. Markers used in cell-types annotation. FIG. 7A, Dot plot of common markers for 4 cell lines. FIGS. 7B-7M, Scatter plots to show important markers used in cell-types annotations with the scONE-seq dataset (top) and Smart-seq2 dataset (bottom). FIGS. 7N-7S, Scatter plots to show important markers used in cell-types annotations with the scONE-seq dataset (top) and Smart-seq2 dataset (bottom). FIGS. 7T-7W, scONE-seq captured Treg cells (FOXP3+, CCR4+). FIGS. 7X-7AA, Non-polyA genes like PZP and SESN3 are captured with scONE-seq data.

FIGS. 8A-8E. Copy number profiles of 3 NPC43 clones. Dots plot with normalized counts across the human genome and solid line plots for corresponding estimated integer copy number. Amplification regions are highlighted with red; deletion regions are highlighted with light blue. Data from the NPC43 cell line establishment state (WGS data, FIG. 8A), the NPC43 C1 clone pseudo bulk data (FIG. 8B, n=20), the NPC43 C2 clone pseudo bulk data (FIG. 8C, n=19), and the NPC43 C3 clone pseudo-bulk data (FIG. 8D, n=69). FIG. 8E, Annotate the clonal information to UMAP with transcriptome data; copy number changes do not impact the gene expression profiles significantly in this case.

FIG. 9. Clinic histological photomicrographs. Representative H&E (first row, 200×) and immunohistochemical stains (2-4 rows, 400×) of the primary tumor, 1^{st} recurrence, and 2^{nd} recurrence of the IDH1-mutant astrocytoma. The primary and recurrent tumors demonstrate similar histologic characteristics featured by pleomorphic hyperchromatic astrocytic cells with brisk mitosis (arrow) (H&E), endothelial proliferation (arrowhead) (H&E), and necrosis (asterisk) (H&E, 2^{nd} recurrence). The tumors show cytoplasmic positivity for IDH1-R132H (second row), loss of ATRX expression (third row), and nuclear positivity for p53 (fourth row).

FIGS. 10A-10H. The genome information of the 2R IDH1-mutant astrocytoma. FIG. 10A, Heatmap showing the normalized counts of the DNA data from scONE-seq profiling of the 2R IDH1-mutant astrocytoma. 4 genome states, normal cell (n=586), 2R clone 1 (n=17), 2R clone 2 (n=20), 2R clone 3 (n=432), are presented to show the CNAs in different cells. FIG. 10B, The FACS sorting plot showing the DAPI intensity density of nuclei from the frozen tumor sample. Most of the cells are diploid. The doubled or even higher DAPI intensity indicates the aneuploid cells with presumed genome duplication. FIG. 10C, Heatmap showing the BAF imputed from scONE-seq DNA data. 4 genome states, normal cell (n=586), 2R clone 1 (n=17), 2R clone 2 (n=20), 2R clone 3 (n=432), are presented to show the BAF in different cells. FIG. 10D, Heatmap showing the mirrored BAF imputed from scONE-seq DNA data. 4 genome states, normal cell (n=586), 2R clone 1 (n=17), 2R clone 2 (n=20), 2R clone 3 (n=432), are presented to show the LOH in different cells. FIG. 10E, Heatmap showing CNAs from WES and scONE-seq clonal pseudo-bulks. The 2R clone 1 and WES inferred P clone 1 both have chromosome 4q deletion. FIGS. 10F-10H, Dots plots showing the chromosome 9 genome states in 3 clones. The CDKN2A homozygous deletion happened in 2R clone 2 and clone 3.

FIGS. 11A-11F. Characterization of 2R clone 1 cells. FIG. 11A, Scatter plots show the expression pattern of 4 key markers to distinguish the 2R clone 1 cells in the scONE-seq RNA dataset. XIST (deletion in clone 3), RFX3 (homozygous deletion in clone 3), ADCY8 and GRIA1 (unique expression in clone 1 compared to normal astrocytes). FIG. 11B, UMAP of 10×Genomics snRNA-seq data validating the cell type composition found using scONE-seq. FIG. 11C, Dot plot with 10×Genomics snRNA-seq shows markers used to annotate cell types. FIG. 11D, Scatter plots show the expression pattern of 4 key markers to distinguish 2R clone 1 cells in the 10×Genomics snRNA-seq dataset. XIST (deletion in clone 3), RFX3 (homozygous deletion in clone 3), ADCY8 and GRIA1 (unique expression in clone 1 compared to normal astrocytes). FIGS. 11E-11F, Violin plots show the expression pattern of the markers selected for immunostaining validation, IDH1 and ADCY8.

FIGS. 12A-12D. Analysis of 2R clone 1 distribution and gene expression in the context of the tumor microenvironment. FIG. 12A, Tumor section fluorescence images showing the spatial distribution of the IDH1 (R132H) and ADCY8 positive cells in the FFPE sections (both primary and 2R tumors). Regions with strong ADCY8 signals were circled with the green line. These regions appear to be ‘normal-adjacent’ tissues (low IDH1 (R132H) signals indicated with yellow arrows). FIG. 12B, Scatter plots showing the expression pattern of astrocytic gene APOE and AMPAR subunit GRIA1 co-expression in many 2R clone 1 cells. FIGS. 12C-12D, Heatmaps shows the potential communication strength between cell types in different secretory pathways. Each row indicates a certain secretory pathway. Left shows the potential to send out certain pathway ligands; Right heatmap shows the potential to receive certain pathway signals with receptors expression. 2R clone 1 cells were predicted to be an important source of the TGFβ ligand source.

FIGS. 13A-13G Data generated using the subject method on nasopharyngeal carcinoma cell (NPC) lines that harbor Epstein-Barr virus. RNA part generated using the subject method shows heterogeneous expression of viral RNA (FIG. 13A: EBV, FIG. 13B: EBER2, FIG. 13C: BWRF1, FIG. 13D: MMP1, FIG. 13E: KRT13, FIG. 13F: CD24) in different NPCs, compared with some endogenous heterogeneous human genes' expression. FIG. 13G: correlation between EBV genome abundance and EBV mRNA abundance in each single cell; general correlation is strong between the two, but some cells show high viral mRNA content while having low genome content, and vice versa, which demonstrates the applicability of the subject method for probing cellular heterogeneity in the context of studying virus activity within host cells.

### BRIEF DESCRIPTION OF THE SEQUENCES

SEQ ID NO: 1: Exemplary Adapter for RNA sequence

SEQ ID NO: 2: Exemplary Amplification primer

SEQ ID NO: 3: Annealing sequence

SEQ ID NO: 4: One-Tn5 Exemplary Adapter for DNA sequence

SEQ ID NO: 5: Exemplary Adapter for RNA sequence

SEQ ID NO: 6: Exemplary Adapter for RNA sequence

SEQ ID NO: 7: Exemplary amplification primer

SEQ ID NO: 8: Mosaic Sequence

SEQ ID NO: 9: Read1-Tn5 sequence/Read 1 primer

SEQ ID NO: 10: I7 index primer

SEQ ID NO: 11: I5 index primer

SEQ ID NO: 12: Read 2 primer

### DETAILED DISCLOSURE OF THE INVENTION

### Selected Definitions

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” include the phrases “consisting essentially of,” “consists essentially of” “consisting,” and “consists.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, 32P and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., a barcode. A barcode is a sequence of about 4 to about 10 nucleotides or about 5 to about 8 nucleotides that are used to distinguish between different samples during sequence analysis.

As used herein, the term “positive,” when referring to a result or signal, indicates the presence of an analyte or item that is being detected in a sample. The term “negative,” when referring to a result or signal, indicates the absence of an analyte or item that is being detected in a sample. Positive and negative are typically determined by comparison to at least one control, e.g., a threshold level that is required for a sample to be determined positive, or a negative control (e.g., a known blank). A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters, and will understand which controls are valuable in a given situation and be able to analyze data based on comparisons to control values. Controls are also valuable for determining the significance of data. For example, if values for a given parameter are variable in controls, variation in test samples will not be considered as significant.

As used herein, a “calibration control” is similar to a positive control, in that it includes a known amount of a known analyte. In the case of a PCR assay, the calibration control can be designed to include known amounts of multiple known analytes. The amount of analyte(s) in the calibration control can be set at a minimum cut-off amount, e.g., so that a higher amount will be considered “positive” for the analyte(s), while a lower amount will be considered “negative” for the analyte(s). In some cases, multilevel calibration controls can be used, so that a range of analyte amounts can be more accurately determined. For example, an assay can include calibration controls at known low and high amounts, or known minimal, intermediate, and maximal amounts.

As used herein, “subject,” “patient,” “individual” and grammatical equivalents thereof are used interchangeably and refer to, except where indicated, mammals, such as humans and non-human primates, as well as rabbits, felines, canines, rats, mice, squirrels, goats, pigs, deer, and other mammalian species. The term does not necessarily indicate that the subject has been diagnosed with a particular disease, but typically refers to an individual under medical or veterinary supervision. A patient can be an individual that is seeking treatment, monitoring, adjustment or modification of an existing therapeutic regimen, etc.

The term “biological sample” or “sample from a subject” encompasses a variety of sample types obtained from an organism. The term encompasses bodily fluids such as blood, blood components, saliva, nasal mucous, serum, plasma, cerebrospinal fluid (CSF), urine and other liquid samples of biological origin, solid tissue biopsy, tumor, tissue cultures, or supernatant taken from cultured patient cells. In the context of the present disclosure, the biological sample is typically a cell or nucleus sample with detectable amounts of nucleic acids. The biological sample can be processed prior to assay, e.g., to lyse cells. The term encompasses samples that have been manipulated after their procurement, such as by treatment with reagents, solubilization, sedimentation, or enrichment for certain components.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

As used herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a nucleotide probe used in the method of this invention has at least 70% sequence identity, preferably 80%, 85%, 90%, 91%, 92%, 93%, 94%, 95%, 96%, 97%, 98%, 99%, or 100% identity, to a target sequence or complementary sequence thereof), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

As used herein, the term “multiplexing” refers to a process in which multiple samples or multiple types of biomolecules are pooled together for signal readout and processing, such as, for example, mixing sequences from multiple single cells into one pool for sequence amplification or sequencing together; or, in another example, generating a mixture of sequences derived from genomic DNA and RNA for amplification or sequencing together.

As used herein, the term “demultiplexing” refers to a process in which of converting the signal/readout from multiple sample origins into separate signals/readouts, which can be performed after a multiplexing experiment is conducted in order to recover the sample-specific information from the pooled/multiplexed readout. For example, converting the sequencing information containing sequences from multiple single cells into sequences derived from each original single cell, possibly based on barcode/adapter/index tags on these sequences that identify their cell-of-origin. In another example, converting the sequence information from the co-amplification of DNA and RNA from a single cell, into sequences derived from DNA of that cell separate from the sequences derived from RNA of that cell, possibly based on barcode/adapter/index tags on these sequences that identify their original biomolecule type.

As used herein, the term “adapter” refers to a nucleic acid component, generally DNA, which provides a means of addressing a nucleic acid fragment to which it is subsequently joined. For example, in certain embodiments, an adapter comprises a nucleotide sequence that permits identification, recognition, and/or molecular or biochemical manipulation of the DNA to which the adapter is attached (e.g., by providing a site for annealing an oligonucleotide, such as a primer for extension by a DNA polymerase, or an oligonucleotide for capture or for a ligation reaction). Adapters may be or include a region that is an indexing/barcoding sequence used to identify the sample source (e.g., cell or tissue) from which each nucleic acid originated to allow multiplexing of molecules from different sample sources for high-throughput amplification and/or sequencing. Alternatively or additionally, indexing/barcoding sequences can be used to distinguish those nucleic acids derived from DNA from nucleic acids derived from RNA (e.g., cDNA) to allow pooling of DNA and RNA from the same sample for high-throughput amplification and/or sequencing. For example, a “DNA-specific barcode” can be used to identify sequences originating from genomic DNA molecules and an “RNA-specific barcode” can be used to identify sequences originating from RNA molecules. Adapters can be added to a nucleic acid, for example, by various enzymatic methods including but not limited to reverse transcription, ligation, tagmentation, PCR, or any combination thereof

### Adapter and Primer Design and Detection

In certain embodiments, the subject invention provides an isolated synthetic nucleic acid adapter in which the adapter can be recognized by the transposase. Transposases can act in complex with specific DNA sequences or adapters, which can form stable complexes with transposases and thus render them active. The adapters can comprise transposase recognition sequences found in nature, or they also can be modified native sequences.

In certain embodiments, the adapter can comprise one or more double-stranded DNA (dsDNA) or single-stranded DNA (ssDNA) sequences. The sequences can be included to allow attachment of generated DNA fragments to sequencing chips, such as Illumina chips, and allow identification of the source of the target DNA and RNA. The adapter can be designed for other types of sequencing, including for example Ion Torrent and DNBSEQ. The adapter can comprise at least one of the following: an amplification primer sequence, a DNA-specific or RNA-specific barcode, a Seq-1 primer, an annealing sequence, and a mosaic. In certain embodiments, the DNA-specific barcode and RNA-specific barcode can be used to differentiate between DNA sequences and RNA sequences in a single sample. In certain embodiments, the adapter can comprise the following exemplary sequence: GTCTCGTGGGCTCGG ATCGT NNNNN TTTTTTTTTTTTTTTTTTTTVN (SEQ ID NO: 1). In certain embodiments, a plurality of adapters, each of which with distinct sequences, can be added to a reaction mixture. In preferred embodiments, the adapter of SEQ ID NO: 1 can be added into a reaction mixture with additional adapters to, for example, achieve capture of non-polyadenylated RNA from the sample, and such an adapter can comprise the following two exemplary classes of sequences: GTCTCGTGGGCTCGG ATCGT NNNNN GGG HN (SEQ ID NO: 5), and GTCTCGTGGGCTCGG ATCGT NNNNN TTT VN (SEQ ID NO: 6). In certain embodiments, an exemplary amplification primer in the adapter is GTCTCGTGGGCTCGG (SEQ ID NO: 2) or GATGTGTGGAGGTCTCGTGGGCTCGG (SEQ ID NO: 7), which is complementary to the Illumina sequencing primer Seq-1. In preferred embodiments, the PCR primer sequence is a sequence within an adapter sequence that is shared between a plurality of adapters that permits simultaneous amplification of nucleotide sequences derived from a sample, including, for example, both DNA and RNA sequences. In certain embodiments, an exemplary RNA barcode in the adapter is ATCGT. In certain embodiments, an exemplary DNA barcode in the adapter is TCATG. In certain embodiments, the UMI in the adapter can be any sequence of at least 3, 4, 5, 6, 7, 8, 9, 10, 11, 12, 13, 14, 15, 16, 17, 18, 19, 20 or more nucleotides, preferably 5 nucleotides in length (i.e., 25), and can be used to uniquely tag each DNA and RNA molecule. In certain embodiments, an exemplary annealing sequence in the adapter: TTTTTTTTTTTTTTTTTTTTVN (SEQ ID NO: 3). In certain embodiments, the Tn5 transposase can recognize specific sequences to form a complex. The mosaic sequence can be the recognition sequence for Tn5, such as, for example exemplary sequence: [phos]CTGTCTCTTATACACATCT (SEQ ID NO: 8). The Illumina platform can design a sequence (Read 1 sequencing primer) to perform sequencing. The Read 1 sequencing primer can include two parts, the mosaic and Seq-1 sequence. In certain embodiments, an adapter or plurality of adapters can be used to tag DNA with a DNA recognition barcode. Additional, the adapter or plurality of adapter used to tag DNA can be used for assembly with Tn5 during the initial tagmentation step. In certain embodiments, the adapter has the sequence: GTCTCGTGGGCTCGG TCATG NNNNN AGATGTGTATAAGAGACAG (One-Tn5) (SEQ ID NO: 4). In preferred embodiments, the co-amplification of DNA and RNA are achieved by performing PCR using a common primer that is shared between the tagged DNA and RNA molecules.

In certain embodiments, an adapter that is complementary to the Tn5 adapter used for tagging DNA, and also contains Read1 sequencing primer region, can be added to the cDNA and gDNA library created by amplifying the tagged DNA and RNA molecules. During a second Tn5 library construction step, this adapter can be assembled with the second round of Tn5. The mosaic sequence can be shared for the two Tn5 steps. In certain embodiments, the sequence of the adapter can be TCGTCGGCAGCGTC AGATGTGTATAAGAGACAG (Read1-Tn5) (SEQ ID NO: 9).

In certain embodiments, one or more adapters that contain primer sequences that amplify a nucleic acid region (or amplicon) of at least 200 bp, about 200 bp to about 6000 bp, about 200 bp to about 4000 bp, about 200 bp to about 3000 bp, about 200 bp to about 2000 bp, about 200 bp to about 1000 bp, about 200 bp to about 750 bp, about 200 bp to about 500 bp, about 200 bp to about 1000 bp, about 200 bp to about 500 bp, or about 300 to about 500 bp is provided by the subject invention. The primer for the amplification reactions can be designed according to known algorithms or by a skilled artisan. For example, algorithms implemented in commercially available or custom software can be used to design primers for amplifying the target sequences based on the complementarity and stringency of said primers to the target region. Stringency refers to hybridization conditions chosen to optimize binding of polynucleotide sequences with different degrees of complementarity. Stringency is affected by factors such as temperature, salt conditions, the presence of organic solvents in the hybridization mixtures, and the lengths and base compositions of the sequences to be hybridized and the extent of base mismatching, and the combination of parameters is more important than the absolute measure of any one factor.

Typically, the primer sequences can be at least 12 bases, more often about 15, about 18, about 20, about 21, about 22, about 23, about 24, about 25, about 26, about 27, about 28, about 29, about 30, or more base pairs in length. In preferred embodiments, the primer sequence is about 26 base pairs in length. Primers are typically designed so that all primers participating in a particular reaction have melting temperatures that are within 5° C., and most preferably within 2° C. of each other. Primers are further designed to avoid priming on themselves or each other. Primer and/or adapter concentration should be sufficient to bind to the amount of target sequences that are amplified so as to provide an accurate assessment of the quantity of amplified sequence. Those of skill in the art will recognize that the amount of concentration of primer and/or adapter will vary according to the binding affinity of the primers as well as the quantity of sequence to be bound.

In certain embodiments, adapters can be designed to hybridize to a nucleic acid sequence, or portions thereof. In certain embodiments, the complementary nucleotide segment of the primer is 1, 2, 3, 4, 5, 6, 7, 8, 9, 10, 11, 12, 13, 14, 15, 16, 17, 18, 19, 20, 21, 22, 23, 24, 25, 30, 40, 50, or 100 base pairs long, or longer. In preferred embodiments, the complementary nucleotide segment of the adapters is about 15 to about 60 base pairs, preferably about 16 to about 50 base pairs, more preferably about 17 to about 40 base pairs, more preferably about 17 to about 35 base pairs, more preferably about 18 to about 25 base pairs. In certain embodiments, the primers can be 100% complementary to a target sequence or at least 70%, 75%, 80%, 85%, 90%, 91%, 92%, 93%, 94%, 95%, 96%, 97%, 98%, or 99% sequence complementarity. In certain embodiments, the sequence of the primer can also have multiple possible alternative nucleotides represented by the IUPAC notation of, for example, R, Y, S, W, K, M, B, D, H, V, N, or a gap (“-” or “.”) nucleotide. In certain embodiments, adapters can be designed to ligate to a nucleic acid sequence, or portions thereof.

### Method of Co-Amplification of DNA and RNA

The invention provides methods for co-profiling of DNA-encoded and RNA-encoded information from a single cell. The method can use a series of molecular biology reactions that act preferentially on DNA and RNA sequentially to tag the DNA and RNA with different nucleic acid barcodes respectively prior to single cell DNA/RNA co-amplification.

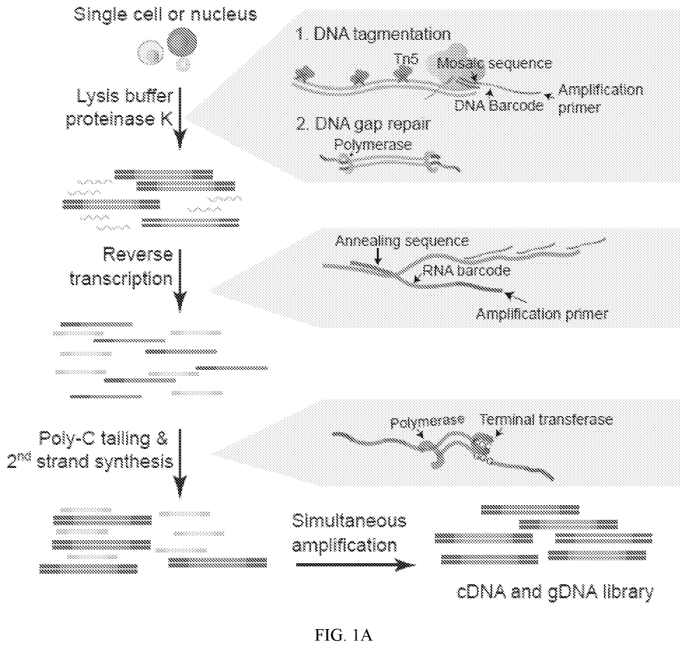

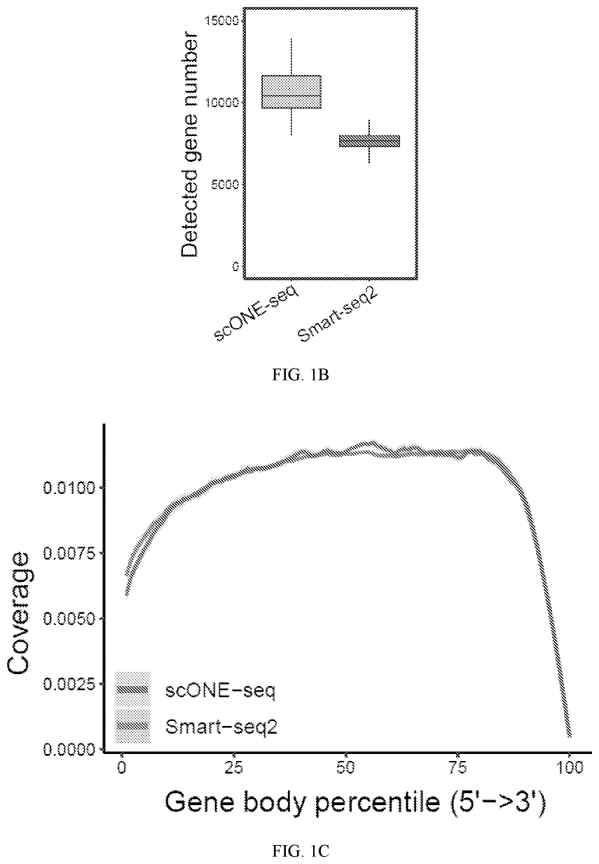

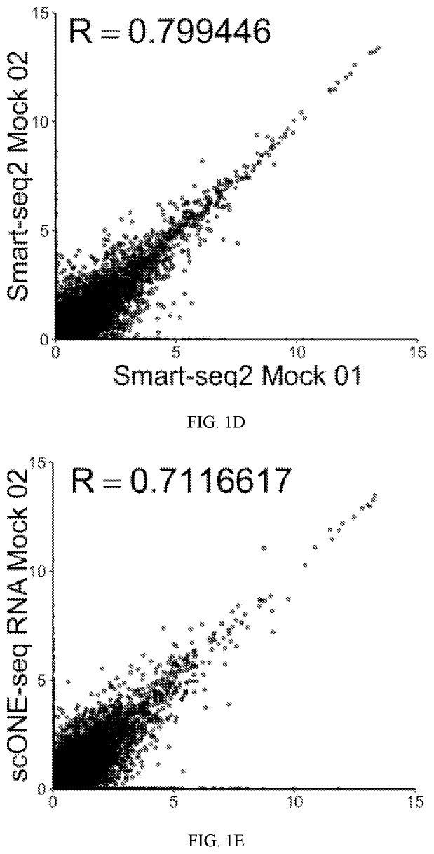

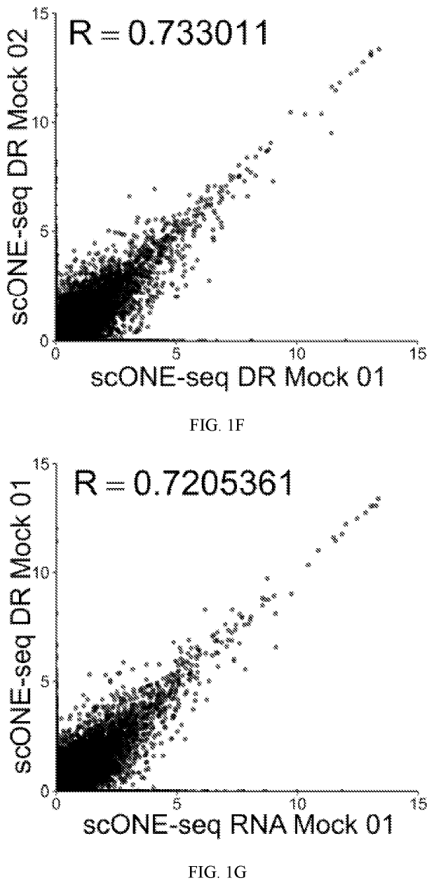

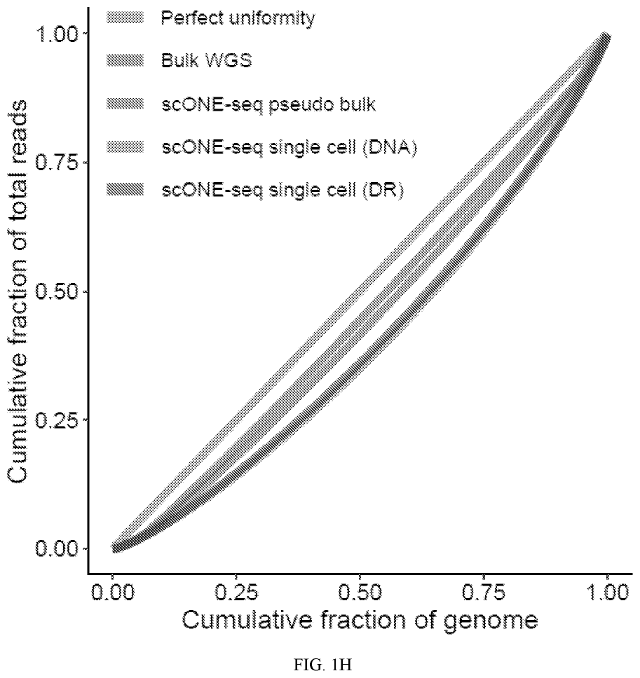

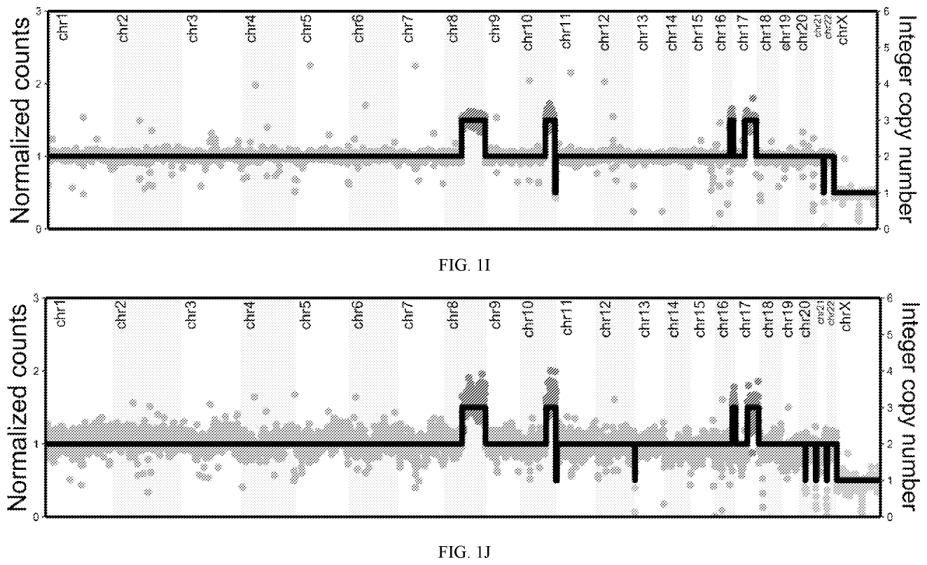

To achieve single cell genome and transcriptome parallel sequencing, we devised the method to co-amplify RNA with DNA in a reaction (FIG. 1A). In a specific implementation of this method, a sample provided can optionally undergo cell lysis and/or nucleic acid extraction. In certain embodiments, the sample containing nucleic acids can undergo Tn5 transposon-based DNA fragmentation and barcode labelling, resulting in fragmented DNA (fDNA). In alternative embodiments, other transposases can be used, such as, for example muA or Tn7. In preferred embodiments, fragmentation can occur simultaneously with barcode labelling using a single enzyme, such as, with Tn5 transposase. In alternative embodiments, DNA fragmentation can occur first followed by barcode labeling. In certain embodiments, subsequent reverse transcription can transform RNA to cDNA and labels the cDNA with a different barcode than the DNA. Each nucleic acid molecule can also be tagged with a unique molecular identified (UMI) which can later be used to distinguish each amplicon for deduplication purposes, to enhance the data quality. In certain embodiments, the 3′ tailing strategy can be used to convert cDNA fragments to an amplification ready format. In certain embodiments, fDNA and cDNA can be amplified simultaneously.

In certain embodiments, following co-amplification, PCR amplified fDNA and cDNA can be either shortened for sequencing or the fragments can be used directly for sequencing. These fragments can be length around about 2000 to about 6000 base pairs in length (fDNA) and about 200 to about 2000 base pairs in length (cDNA). In certain embodiments, next-generation sequencing (NGS) requires shorter nucleotide lengths than other types of sequencing. Therefore, to make the sequences appropriate lengths for short read sequencing in platforms, such as, for example, the Illumina platform, a second Tn5 tagmentation can be used to fragment the co-amplified fDNA and cDNA. This second tagmentation can insert adapters that contain a sequencing primer, such as, for example, the Read1 primer (SEQ ID NO: 9) (Illumina-specific), so that the final products can be dsDNA fragments of about 400 to about 800 base pairs in length and contain a sequencing primer, such as, for example, Read1 (SEQ ID NO: 9) and Read2 (SEQ ID NO: 12). In certain embodiments, any further specific sequencing primers can be added in more PCR steps after this step.

In certain embodiments, a “sample index” sequence is included in the DNA barcode adapter and RNA barcode adapter, where the same sample has the same sample index for both the DNA and RNA molecules. This allows the co-amplified library from multiple different single cells to be pooled and sequenced simultaneously in a multiplexed fashion.

Any high-throughput method for sequencing can be used in the practice of the invention. DNA sequencing approaches include, but are not limited to: dideoxy sequencing reactions using labeled terminators (the Sanger method) in various formats, sequencing by synthesis, pyrosequencing, real time monitoring of the incorporation of labeled nucleotides during a polymerization step, high-throughput single-molecule sequencing.

After the co-amplified library is sequenced, data processing can be used to filter and separate reads from DNA/RNA. In certain embodiments, the sequenced library of reads can be aligned to the known “DNA barcode” and “RNA barcode” to determine whether any given read is from DNA or RNA, thus separating the two. In certain embodiments, the “fastp”, “seqkit”, and/or “seqtk” programs can be used to perform the separation. After separating the DNA from the RNA, the known adapter sequences can be trimmed/removed using programs such as Cutadapt, the FASTX toolkit, and others. In certain embodiments, the separated DNA/RNA reads can be aligned to a reference sequence dataset, either computationally or manually; for example, the DNA reads aligned to an organism's genome reference and the RNA reads aligned to an organism's transcriptome reference. Following reads preprocessing, the DNA and RNA data can be analyzed separately using standard single-cell analysis computational pipelines, such as, for example, Seurat, Gingko, or CHISEL to perform the data analysis and visualization. In other embodiments, the separated DNA/RNA reads can be assembled from short reads into longer reads or contigs representing a longer contiguous nucleic acid sequence, for the purpose of de novo sequence or genome/transcriptome assembly. The assembly can be done using a pipeline composed of several different programs such as Spades (short reads), Canu (long reads), Velvet, followed by BUSCO, and the like.

In certain embodiments, fragmentation of DNA can be achieved by enzymatic digestion or physical methods such as sonication, nebulization or hydrodynamic shearing. The fragmentation of the DNA can be achieved using a Tn5 transposase, as described in Zahn, H., Steif, A., Laks, E. et al. Scalable whole-genome single-cell library preparation without preamplification. Nat Methods 14, 167-173 (2017). See worldwide website: doi.org/10.1038/nmeth.4140.

The method provides a DNA/RNA barcoding strategy, which tags the DNA and RNA with different nucleic acid barcodes prior to single cell DNA/cDNA co-amplification. Amplification adapters are also added and used to co-amplify the DNA and reverse transcribed cDNA with the same primer sets, generating the sequencing library. In certain embodiments, the Tn5 transposase can also ligate an adapter to a cDNA nucleic acid sequence and/or DNA nucleic acid sequence. After sequencing, DNA and RNA reads can be computationally distinguished by demultiplexing of the barcode.

In certain embodiments, the cell from a sample can be added to a lysis buffer that contains components such as, for example, SDS, Triton X-100, or Tween-20 before amplification and/or reverse transcription of the nucleic acids targets; optionally the lysis buffer contains nuclease inhibitors like RNAse inhibitor. The cell from the sample can also be added to a buffer containing protease, such as, for example proteinase K or Thermolabile Proteinase K (New England Biolabs, Ipswich MA), before amplification and/or reverse transcription of the nucleic acids targets.

In certain embodiments, the detection of the at least one single-stranded or double stranded nucleic acid is carried out in an enzyme-based nucleic acid amplification method.

The expression “enzyme-based nucleic acid amplification method” relates to any method wherein enzyme-catalyzed nucleic acid synthesis occurs.

Such an enzyme-based nucleic acid amplification method can be preferentially selected from the group constituted of Polymerase Chain Reaction (PCR), notably encompassing all PCR based methods known in the art, such as reverse transcriptase PCR (RT-PCR), simplex and multiplex PCR, real time PCR, end-point PCR, quantitative or qualitative PCR and combinations thereof. These enzyme-based nucleic acid amplification method are well known to the man skilled in the art and are notably described in Saiki et al. (1988) Science 239:487, EP 200 362 and EP 201 184 (PCR); Fahy et al. (1991) PCR Meth. Appl. 1:25-33 (3SR, Self-Sustained Sequence Replication); EP 329 822 (NASBA, Nucleic Acid Sequence-Based Amplification); U.S. Pat. No. 5,399,491 (TMA, Transcription Mediated Amplification), Walker et al. (1992) Proc. Natl. Acad. Sci. USA 89:392-396 (SDA, Strand Displacement Amplification); EP 0 320 308 (LCR, Ligase Chain Reaction); Bustin & Mueller (2005) Clin. Sci. (London) 109:365-379 (real-time Reverse-Transcription PCR).

In some embodiments, the enzyme-based nucleic acid amplification method is selected from the group consisting of Polymerase Chain Reaction (PCR) and Reverse-Transcriptase-PCR (RT-PCR), multiplex PCR or RT-PCR and real time PCR or RT-PCR. In other embodiments, the enzyme-based nucleic acid amplification method is a real time, optionally multiplex, PCR, quantitative PCR or RT-PCR method.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Successful PCR amplification requires high yield, high selectivity, and a controlled reaction rate at each step. Yield, selectivity, and reaction rate generally depend on the temperature, and optimal temperatures depend on the composition and length of the polynucleotide, enzymes and other components in the reaction system. In addition, different temperatures may be optimal for different steps. Optimal reaction conditions may vary, depending on the target sequence and the composition of the primer. Thermal cyclers such as, for example, real-time PCR systems provide the necessary control of reaction conditions to optimize the PCR process for a particular assay. For instance, a real-time PCR system may be programmed by selecting temperatures to be maintained, time durations for each cycle, number of cycles, and the like. In some embodiments, temperature gradients may be programmed so that different sample wells may be maintained at different temperatures, and so on.

In certain embodiments, the target nucleic acid sequence can be RNA and DNA. RNA or DNA can be artificially synthesized or isolated from natural sources. In some embodiments, the RNA target nucleic acid sequence can be a ribonucleic acid such as RNA, mRNA, piRNA, tRNA, rRNA, ncRNA, gRNA, shRNA, siRNA, snRNA, miRNA and snoRNA More preferably the DNA or RNA is biologically active or encodes a biologically active polypeptide. The DNA or RNA template can also be present in any useful amount.

Reverse transcriptases useful in the present invention can be any polymerase that exhibits reverse transcriptase activity. Several reverse transcriptases are known in the art and are commercially available (e.g., from Bio-Rad Laboratories, Inc., Hercules, CA; Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the reverse transcriptase can be Avian Myeloblastosis Virus reverse transcriptase (AMV-RT), Moloney Murine Leukemia Virus reverse transcriptase (M-MLV-RT), Human Immunovirus reverse transcriptase (HIV-RT), EIAV-RT, RAV2-RT, C. hydrogenoformans DNA Polymerase, rTth DNA polymerase, SUPERSCRIPT I, SUPERSCRIPT II, SUPERSCRIPT III, and mutants, variants and derivatives thereof. It is to be understood that a variety of reverse transcriptases can be used in the present invention, including reverse transcriptases not specifically disclosed above, without departing from the scope or preferred embodiments disclosed herein.

DNA polymerases useful in the present invention can be any polymerase capable of replicating a DNA molecule. Preferred DNA polymerases are thermostable polymerases and polymerases that have exonuclease activity, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothemophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof. Preferred DNA polymerases have strand displacement activity; however, a polymerase with strand displacement activity is not required and other methods known in the art of displacing nucleotide strands can be used in the subject invention, such as, for example, heating the nucleotide strands. In preferred embodiments, a high fidelity polymerase can be used. In certain embodiments, a single polymerase can be used or two or more distinct polymerases can be used. In certain embodiments, the polymerase is KAPA HiFi (Roche, Basel, Switzerland)

Many DNA polymerases are known in the art and are commercially available (e.g., from Bio-Rad Laboratories, Inc., Hercules, CA; Boehringer Mannheim Corp., Indianapolis, Ind.; Life Technologies, Inc., Rockville, Md; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif). In some embodiments, the DNA polymerase can be Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, and active mutants, variants and derivatives thereof. It is to be understood that a variety of DNA polymerases can be used in the present invention, including DNA polymerases not specifically disclosed above, without departing from the scope or preferred embodiments thereof.

In certain embodiments, the proportion of DNA or RNA in the final sequencing library can be adjusted by changing the Tn5 concentration in the initial reaction mixture.

In a preferred embodiment, the reactions according to the invention can also contain further reagents suitable for a PCR step. Such reagents are known to those skilled in the art, and include water, like nuclease-free water, RNase free water, DNAse-free water, PCR-grade water; salts, like magnesium, magnesium chloride, potassium; buffers such as Tris; enzymes; nucleotides like deoxynucleotides, dideoxunucleotides, dNTPs, dATP, dTTP, dCTP, dGTP, dUTP and modified nucleotides such as deaza-, locked nucleic acid, and peptide nucleic acid; other reagents, like DTT and/or RNase inhibitors; and polynucleotides like polyT and polydT.

The methods of the subject invention can be easy to use and simple to adopt, requiring no additional or specialized equipment beyond what is available in standard biology laboratories and using standard wet lab operating procedures. The methods can be automatable and scalable, as it only requires standard pipetting steps and thus can be adapted to use liquid handling robots for high-throughput applications. In certain embodiments, the methods of the subject invention can be comparable in accuracy and sensitivity to existing single-cell profiling methods that profile only DNA or RNA from a single cell. In certain embodiments, the methods of the subject invention can be superior in accuracy and sensitivity to existing single-cell profiling methods that profile both DNA and RNA from a single cell. The subject method, scONE-seq, can enable numerous previously intractable single cell multi-omic experiments, and lead to new discoveries in the life sciences.

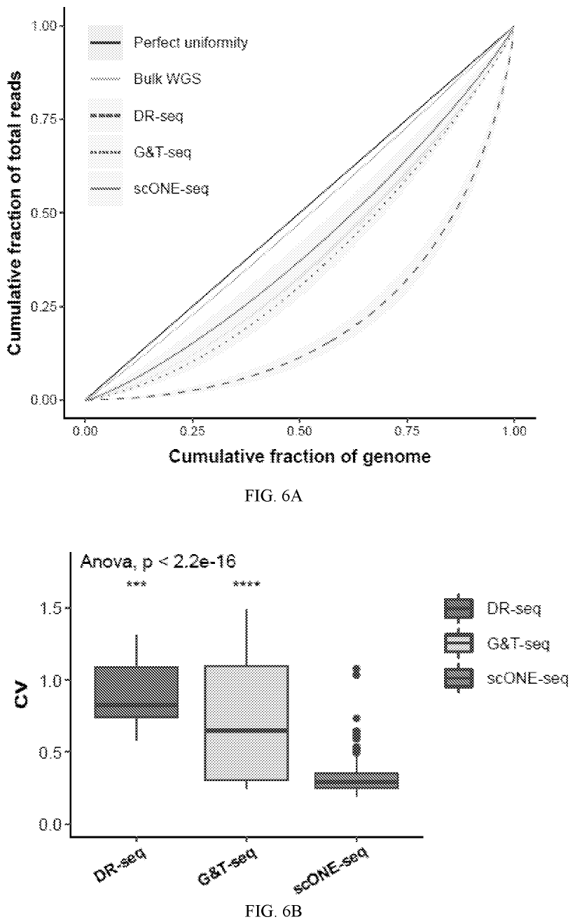

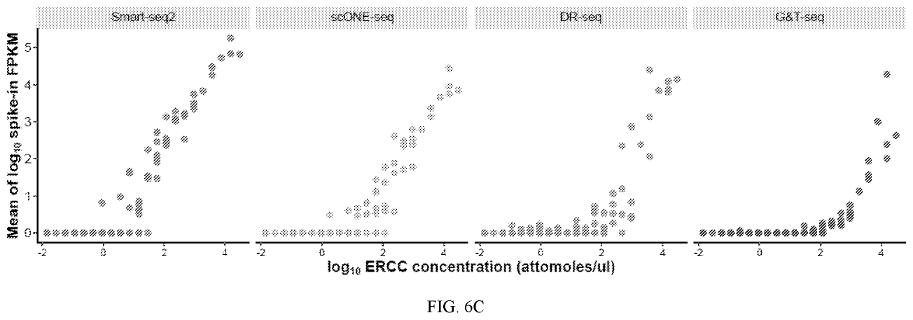

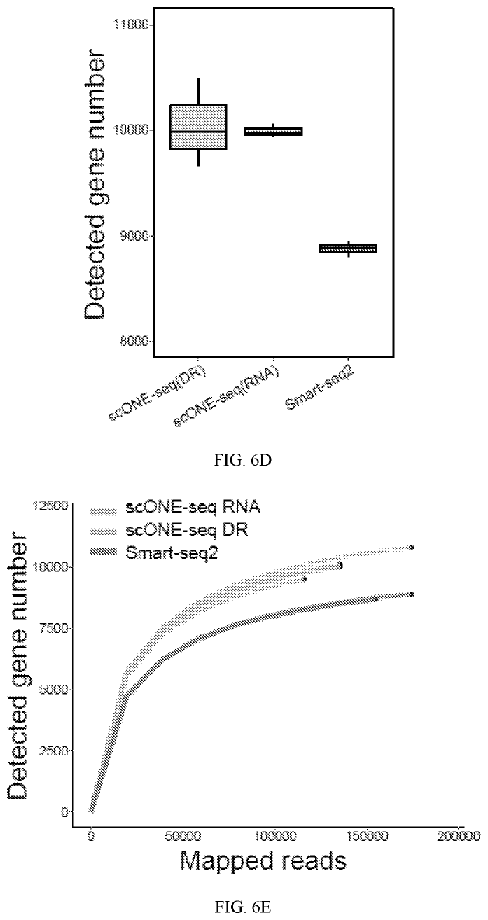

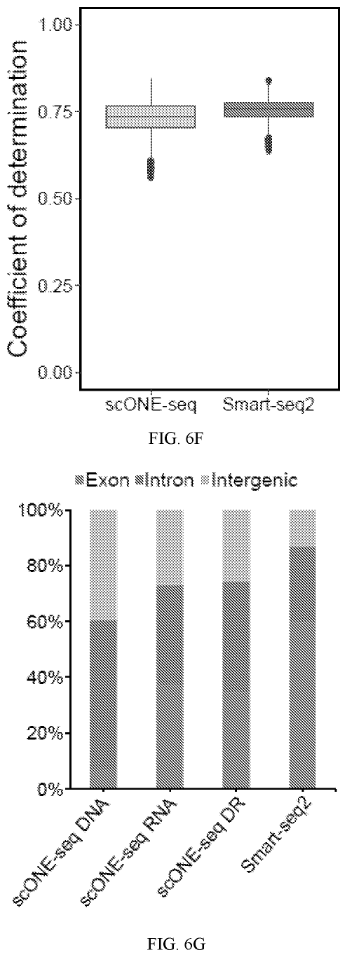

In Table 1, some of the key advantages of scONE-seq compared to G&T-seq and DR-seq. From the published data, DR-seq can suffer from GC-bias in the DNA amplification, and the overall amplification uniformity is worse (FIG. 6A-6B), whereas G&T-seq has reasonably good single-cell whole genome sequencing data, but due to the numerous washing steps required to release non-specific DNA from the RNA capture beads, the sensitivity of transcript detection appears to be lowered (FIG. 6C). scONE-seq can have higher overall single-cell amplification success rate. In certain embodiments, only about at least 1 million reads per sample, including a cell or nucleus, can be used to sequence and to obtain sufficient depth for both RNA and DNA to perform clonal analysis and cell-type classification. From the published data, DR-seq required almost 10-fold more sequencing depth to achieve similar coverage. This represents a substantial cost savings in sequencing by scONE-seq.

The time required for each protocol was estimated from published versions. Due to having only one reaction for each cell, scONE-seq can take at least about 8 hours per plate, and can use a single purification step at the very end. Overall, scONE-seq produces better data with less experimental time and lower cost.

TABLE 1

| | G&T-seq | DR-seq | |
|---|---|---|---|
| | (Macauley et al. | (Dey et al. | |
| | Nature Methods 2015) | Nature Biotechnology 2015) | scONE-seq |
| Approach for | polyT-bead based | Barcoding of RNA with RT | DNA-specific barcoding with Tn5 |
| distinguishing DNA | separation of polyA- | primer to generate barcoded | and RNA-specific barcoding with |
| from RNA | RNA from the DNA for | CDNA, followed by pre- | RT primer in a single tube, |
| | separate DNA and | amplification of cDNA and gDNA | followed by co-amplification with |
| | RNA library | together, and then splitting the | a common primer and joint |
| | construction | entire pool for separate DNA | library construction, with in-silico |
| | | and mRNA library construction | demultiplexing of DNA and RNA |
| Can be used for total | No | No | Yes |
| RNA-seq? | | | |
| Single cell | 75.6% | 70% | 90% |
| amplification success | | | |
| rate (both WGS and | | | |
| RNA pass QC) | | | |
| % GC of DNA reads | 40% (normal) | 50% (biased) | 40% (normal) |
| Time needed from cell | 14 h for one 96 plate | 24 h for one 96 plate | 8 h for one 96 plate |
| to pre-amplified library | | (incl. 13 h IVT incubation time) | |
| Labor demand | Requires 3 purification steps | Requires 2 purification steps per | Only 1 purification step per 96 |
| | per 96 plate; beads-based | 96 plate; requires repeated | plate at the very end |
| | separation step needs | addition of enzymes between | |
| | to be performed | every cycle of quasilinear | |
| | manually with care | amplification for 7 cycles total | |
| Library construction | Tagmentation based | DNA library is prepared with a | Tagmentation based library |
| | library construction | sonication-based method | construction |
| Automation capability | Can be automated | Difficult to automate effectively | Can be automated with a |
| | with a pipetting robot | due to the repeated enzyme | standard liquid handler or a |
| | | adding step between cycles | pipetting robot |

In certain embodiments, the RNA and DNA from a single sample need not be physically separated during the reaction, and can still be differentially tagged within a single reaction compartment. In certain embodiments, the subject method achieves simultaneous tagging and amplification of DNA and RNA in a one-container reaction. In certain embodiments, the subject methods do not require any specifically designed device (such as a microfluidic chip) to achieve co-profiling of DNA and RNA from the same single cell. In certain embodiments, the subject methods can be automated using robots or other high-throughput platforms, such as, for example, a microfluidic platform. This allows the experiment to the scaled up easily to orders of magnitude higher throughput, which can enable the DNA-RNA co-profiling of previously unattainable orders-of-magnitude number of single cells. The throughput is versatile and easy to control, thereby making the method appropriate for small scale use as well as large scale applications.

### Targets of Nucleotide Detection

In certain embodiments, the methods provided by the subject invention can be used to amplify one or more DNA nucleic acid sequences and one or more cDNA sequences derived from RNA nucleic acid sequences from a single cell or nucleus. In certain embodiments, the methods can be used to amplify nucleic acid sequences of fresh cell samples, such as, for example, peripheral blood mononuclear cells (PBMCs) and cells lines. In certain embodiments, the methods can be used to amplify nucleic acid sequences of nuclei from frozen tissue samples, such as, for example, tumor specimens that have been frozen for years. In certain embodiments, the population of cells can be determined, such as, for example, the cell populations of B-cells, T-cells, and NK cells, based on, for example, the gene expression markers. In certain embodiments, different genome and transcriptome profiles can be determined using the subject methods. In certain embodiments, the RNA sequence can be used to determine gene expression markers. In certain embodiments, the DNA sequences can be used to determine copy number alterations (CNAs).

In certain embodiments, the subject methods can be used to probe virus-host interactions. By co-profiling DNA and RNA from a virus and a host cell, the distribution of the virus can be determined within a host. Furthermore, the virus abundance within the host cell can be correlated with the virus gene expression. Using the virus abundance information, all genes with the virus can be selected and the correlated genes can be analyzed for viral patterns, such as, for example, cells could be separated into virus-rich cells and virus-poor cells. In certain embodiments, the methods can be applicable to subcellular level components such as, for example, single nuclei, which also contains both DNA and RNA. In certain embodiments, the methods can be also applicable to any biomolecule in any context that is tagged with DNA or RNA.

The methods of the invention can be useful for any type of cell. Methods of the invention are applied most straightforwardly to the co-profiling of whole genomes and total transcriptomes from the same single cell. The methods can be used for identifying diseases, such as, for example, cancer, in which the genome and transcriptome reflect different facets of the disease progression. The genome reveals the genomic instability and mutational landscape typically associated with cancer initiation and progression; the transcriptome reflects the cell's functional/molecular identity which could be associated with its stemness, the level of differentiation of the cancer, and inform prognoses for patients. The methods are also particularly useful for studying viral activity within cells, as infected cells harbor viral DNA and RNA in addition to their own endogenous genome and transcriptome, and depending on the type of virus, DNA or RNA, it is useful to interrogate both the DNA and RNA to observe the activity of the virus in the cell and its effects on cellular behavior. In addition to eukaryotic cells and their infecting viruses, this application could also include the interrogation of prokaryotes such as bacteria and their interactions with phage. The methods can also be useful for studying any type of symbiont-host interaction, such as, for example, the interaction of bacteriocytes and their host eukaryotic cell. The method may also be used for de novo genome and transcriptome assembly for an organism. The method may also be generalized to drug screening and discovery.

In certain embodiments, the subject methods can be compatible with frozen tissue samples that have been stored for at least hours, days, months, or years. This feature makes it easier to plan and perform larger-scale clinical multi-omic single-cell studies in two ways: first, by enabling studies on existing biobanked samples, which we have demonstrated herein; second, for studies on new samples, it also removes the burden of having to immediately process tissues from clinical researchers whose priority is patient care.

In certain embodiments, the subject methods, including scONE-seq, can be used on frozen glioblastoma (GBM) tissue. In certain embodiments, the subject methods, including scONE-seq, can be used to observe and characterize the differentiated tumor clones, which supports the idea that tumor clones can produce a differentiation hierarchy7,58,59. The existence of clone 1 was confirmed using both independent 10×Genomics snRNA-seq as well as immunostaining on tissue sections. scRNA-seq-only based cancer studies could underestimate important layers of tumor heterogeneity, and that simultaneous direct DNA measurement could contribute meaningful and informative insight on tumor evolution. Meanwhile, the clonal analysis based on scWGS-only data also ignores the complex interactions within a tumor microenvironment. By deciphering the genetic and phenotypic heterogeneity within the tumor ecosystem with the subject methods, including scONE-seq, we can reveal the interplays of clonal expansion, tumor cells differentiation hierarchy, and tumor microenvironment (TME).

Compared to other scDR-seq methods, the subject methods, including scONE-seq, can have much higher throughput. In certain embodiments, the subject methods, including scONE-seq, also possesses very high scalability. Alternatively, producing scONE-seq and droplet-based single cell data in parallel and then integrating them, is also a useful complementary, multi-omics approach to study cancer with high throughput. Moreover, additional processing can be added to the scONE-seq workflow to enable profiling of more layers of information: to detect chromatin accessibility simultaneously, an additional nuclei tagmentation step⁶⁰⁻⁶² with customized ATAC adaptors could be added before FACS sorting; Similarly, quantitative protein estimation⁶³ could be achieved by using DNA-barcoded antibodies before single-cell sorting steps of the scONE-seq (see Stoeckius, M. et al. Simultaneous epitope and transcriptome measurement in single cells. Nature Methods 14, 865-868 (2017).); and by jointly performing whole-exome capturing or any hybridized target sequencing panels with the standard scONE-seq library, paired high-depth single-cell somatic mutation information could also be integrated into the scONE-seq dataset.

### Materials and Method S

### Single Cell or Single Nucleus Isolation.

HCT116, NPC43, HUVEC, and H9 cells are dissociated with trypsin-EDTA (0.25%) solution (Thermo Fisher, Waltham, MA) and stained with propidium iodide (10 mg/ml, Thermo Fisher) to exclude dead cells.

Fresh whole human blood was taken in the clinic center of the HKUST from a healthy donor. Lymphocytes were isolated via Ficoll-Paque PLUS (GE Healthcare, Chicago, IL) density centrifugation. The red blood cells were removed with 1×Red Blood Cell lysis buffer (Thermo Fisher).

The months-old frozen IDH1-mutant glioblastoma tissue (stored at −80° C.) was obtained from Prince of Wales Hospital. The nuclei isolation protocol is based on previous studies^{64,65}. In brief, the homogenization method was used to prepare nuclei. The homogenization douncer should be cleaned with ethanol, bleach, and RNase-out and then rinsed with NF water. 100 mg frozen tissue was put into pre-chilled glass douncer contained 1 ml of 1× homogenization buffer (5 mM CaCl₂, 3 mM Mg(Ac)₂, 10 mM Tris, 16.7 μM PMSF, 167 μM β-mercaptoethanol, 320 mM sucrose, 0.1 mM EDTA, 0.1% NP40, 1 U/ml RNase inhibitor, 1× Proteinase Inhibitor, pH=7.8). The homogenized suspension was then filtered with a 35 μm cell strainer (Corning, Corning, NY), and nuclei can be spin down at 1000 g, 10 mins, 4° C. Nuclei were resuspended in 3.0 ml low sucrose buffer (320 mM sucrose, 10 mM HEPES, 5 mM CaCl₂, 3 mM Mg(Ac)2, 0.1 mM EDTA, 1 mM DTT, 1U/ml RNase inhibitor, 1× Proteinase Inhibitor, pH=8.0). To remove cell debris, we then put 12.5 ml of density sucrose buffer (1 M sucrose, 10 mM HEPES, 3 mM Mg(Ac)2, 1 mM DTT, pH=8.0) underneath the low sucrose buffer homogenate to centrifuge at 3200 g for 20 min at 4° C. The nuclei were now in a flicking motion and can be stained with DAPI (Thermo Fisher).

Cells or nuclei were then loaded to Aria III flow cytometer (BD Biosciences, Franklin Lakes, NJ) to sort single cells into PCR tubes (96 or 384 PCR plates) containing Lysis buffer. The lysis buffer consisted of 2.5 U/μl RNase Inhibitor (NEB, Ipswich, MA), 0.15% Triton X-100 (Sigma, St. Louis, MO), and 6 μM DTT (Thermo Fisher). The sorted sample can be stored at −80° C. for months.

Generation of scONE-Seq Libraries.

To start the scONE-seq pre-amplification, the proteinase K (Sigma) was used to completely lysis cells or nuclei. Tagmentation reaction was performed to fragment the genome DNA and add the DNA-specific barcode. This reaction includes the following components, 6 mM MgCl₂, 0.5 mM dNTP (NEB), 8.5 mM TAPs-NaOH, 1.5 U/μ1 RNase Inhibitor, 0.05 U KAPA polymerase (Roche), 8% PEG8000, and Tn5 with custom adaptor (GTCTCGTGGGCTCGGTCATG AGATGTGTATAAGAGACAG (SEQ ID NO: 4)) (Novoprotein Suzhou, Jiangsu, China)^{33,37}. The reaction was incubated at 55° C. for 10 mins followed by 72° C. for 10 mins. Then, proteinase K or thermolabile proteinase K (NEB) was used to deactivate the enzyme in the buffer. Thereafter, we performed reverse transcription with the following components, 40 U SuperScript™ III Reverse Transcriptase (Thermo Fisher), 70 mM Tris-HCl, 1.5 U/μ1 RNase Inhibitor, 8 mM MgCl2, 7 μM DTT and 0.15 μM RT primers (GTCTCGTGGGCTCGGATCG TTTTTTTTTTTTTTTTTTTTVN (SEQ ID NO: 1); GTCTCGTGGGCTCGGATCGTNNNNNGGGHN (SEQ ID NO: 5); GTCTCGTGGGCTCGGATCGTTTTVN (SEQ ID NO: 6)). Reverse transcription was carried out at 12° C. for 12 sec followed gradient increasing to of 50° C. for 50 min and 55° C. for 50 min. Subsequently, the residual primers and RNA were removed with thermolabile EXO I (NEB), RNase If (NEB), and RNase H (NEB). Then, the terminal transferase (NEB) was used to add the C-tail to cDNA fragments. This reaction was performed at 37° C. for 5 mins and the enzyme was immediately deactivated with thermolabile proteinase K. Second strand synthesis was then performed by adding, 0.3 μM 3′ adaptor (GTCTCGTGGGCTCGGATCGTNNNNNGGGHN (SEQ ID NO: 5)), 1 μl KAPA HIFI Fidelity Buffer (5×), 0.7 mM (NH₄)₂SO₄, and 0.1 μl KAPA Polymerase. The reaction was incubated at 72° C. for 5 min; 10 cycles of (1 min at 48° C.; 1 min at 72° C.); and 5 min at 72° C., in a thermal cycler. Additional residual primers removal reaction was performed with Exo I (NEB). Lastly, 14 μl KAPA HotStart ReadyMix (2×), 1.5 mM (NH₄)₂SO₄, 2% DMSO (Thermo Fisher), 1.2 μM amplification primer (GATGTGTGGAGGTCTCGTGGGCTCGG (SEQ ID NO: 7)) was added to amplify DNA and RNA simultaneously. The PCR was performed at 98° C. for 4 min; 18-20 cycles of (20 s at 98° C.; 4.25 min at 72° C.); and 10 min at 72° C., in a thermal cycler.

### Sequencing Libraries Construction.

Pre-amplified samples were purified with Ampure XP beads (Beckman, Brea, CA). Samples were diluted to 0.1 ng/μl and performed tagmentation reaction with the following components, 1× TAPs buffer (50 mM TAPS-NaOH, 25 mM MgCl2, PH=8.0), 8% PEG8000, 0.001 μl Tn5 (TCGTCGGCAGCGTCAGATGTGTATAAGAGACAG) (SEQ ID NO: 9). The reaction was performed at 55° C. for 15 min. Samples were then amplified with Illumina (San Diego, CA) sequencing index primers (Table 2) (Sangon, Shanghai, China) by using KAPA HiFi HotStart Polymerase Kit (Roche, Basel, Switzerland). The enrichment PCR was incubated at 95° C. for 10 min; 10-11 cycles of (20 sat 98° C.; 15 sat 60° C.; 30 s at 72° C.); and 2 min at 72° C., in a thermal cycler. Samples were then pooled and purified with Ampure XP beads. scDASH protocol was then used to remove the abundant ribosome and mitochondrial RNA^{66,67}. Double-size selection can be performed to optimize the library size. The library was then sequenced on Illumina NextSeq500.

The 17 Index primer is a standard Illumina sequence for sequencing (it is only added when the library is ready to be sequenced). The IS Index primer is the equivalent of 17 index primer on the other side of the sequenced read. Table 2 shows a custom version of IS Index primer used by our method to enable sequencing of scONE-seq products using the Illumina platform (SEQ ID NO: 11). A standard Illumina IS Index primer will not work with the subject scONE-seq libraries. This primer is, like 17 Index primer, added directly to the flow cell during sequencing. Read2 sequence is the equivalent of Read1 on the other side of the sequenced read. Here, Read2 is customized to work with scONE-seq co-amplified products. The standard Illumina Read2 would not work.

TABLE 2
Sequencing primers needed to sequence scONE-seq
co-amplified products using Illumina platform

| | |
|---|---|
| Read1 | TCGTCGGCAGCGTC AGATGTGTATAAGAGACAG (SEQ |
| | ID NO: 9) |
| 17 Index | CCGAGCCCACGAGAC CTGTCTCTTATACACATCT (SEQ |
| primer | ID NO: 10) |
| 15 Index | CTGTCTCTTATACACATCT GACGCTGCCGACGA (SEQ |
| primer | ID NO: 11) |
| Read2 | AGATGTGTATAAGAGACAG GTCTCGTGGGCTCGG (SEQ |
| | ID NO: 12) |

### DNA and RNA Data Separation.

Sequencing data was firstly filtered with fastp⁶⁸. Fastq files were then separated into DNA fastq files, RNA fastq files, and Unmatched fastq files with seqkit, seqtk, and bbduk⁶⁹⁻⁷¹. During this process, UMI of the reads was extracted and labeled to fastq files head with fastp⁶⁸.

### DNA Data Analysis.

DNA fastq files were mapped to hg38 (see worldwide website: ncbi.nlm.nih.gov/assembly/GCF_000001405.26/) with BWA mem⁷². To perform UMI-based deduplication, read2 reads in bam files were extracted with samtools⁷³ and deduplicated with umi_tools⁷⁴. The deduplicated read2 reads were used to extract its paired read1 and these paired fastq were then re-aligned to hg38 with BWA mem^{72,75}.

If only performing the counts-based copy number variation analysis, Ginkgo was used to generate the normalized counts⁷⁶. If performing the allele-specific copy number variation analysis, CHISEL was used to generate both allele frequency information^{77,78}. The integer copy number calculation was based on previous studies⁷⁹⁻⁸¹. In this pipeline, the segmentation was performed with copynumber and aCGH⁸².

### RNA Data Analysis.

UMI-based deduplication was also performed with RNA fastq files. The workflow kept the same except replacing the BWA with STAR⁸³. Then, the fastq files can be quantified with Kallisto⁸⁴ (cDNA quantification) or Salmon⁸⁵ (pre-mature RNA quantification) (ref1-2). 10× snRNA-seq data was quantified with kb-python⁸⁶. The expression data were analyzed using Seurat with sctransform pipeline (normalization, dimension reduction, dataset integration, finding clusters, differential gene analysis)⁸⁷⁻⁸⁹. The GBM cellular states scoring was performed following the original paper⁹⁰. RNA-based CNV inferring was performed with copykat⁹¹. The ligand-receptor analysis was performed with CellChat⁹².

### Visualization.

Plots were created using the ggplot2 R package^{93,94}. Heatmaps were created with the ComplexHeatmap package⁹⁵. R Figures were prepared in Inkscape⁹⁶.

### Ihc Analysis.

Slides were obtained from Dr. Danny Chan (Prince of Wales Hospital). Xylene and ethanol were used to remove wax. Antigen retrieval was performed with Sodium Citrate Buffer (Thermo Fisher) at 98° C. for 15 min. IDH1(R132H) antibody (Dionava, 1:50) and ADCY8 antibody (Abcam, 1:200, Cambridge, UK) were added to slides and incubated at 4° C. overnight in a humiliating box. Secondary antibodies (anti-mouse, anti-rabbit, Thermo Fisher) were used to provide the fluorescent signal. Mounting buffer with DAPI (Abcam) was used to stain the nucleus and retain fluorescence. The images were taken with Zeiss Axio Scan.Z1 Slide Scanner (Zeiss, Jena, Germany).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Method of Co-Amplifying RNA and DNA in a Sample

To achieve single-cell genome and transcriptome co-profiling, we devised a workflow to amplify RNA and DNA simultaneously (FIG. 1A). Briefly, to perform scONE-seq, after the sample dissociation, cells or nuclei are sorted into PCR plates containing lysis buffer with a flow cytometer. Plates of sorted single cells can then be processed immediately, or store at−80 for months before processing. To start the single-cell amplification, we first use Tn5 with a custom adaptor to fragment and label the genome or any other DNA within the cells³¹⁻³³. In this step, the amplification adaptor, which includes a 6-nucleotide “DNA barcode” and 6-nucleotide UMI, is added to fragmented DNA (fDNA). Subsequently, we use reverse transcription (RT) to generate cDNA, where the RT primer is comprised of: a priming sequence adapted from that of the MATQ-seq protocol³⁴, a 6-nucleotide “RNA barcode”, and a 6-nucleotide UMI. The RT priming sequence is a modified random oligo and primes to the internal regions of RNA transcripts, thereby enabling detection of full-length transcripts, including non-polyadenylated (non-polyA) RNAs. cDNA 3′ adaptors are added through subsequent poly-C tailing and degenerate PCR³⁵. Once DNA-specific and RNA-specific barcodes have been added, fDNA and cDNA are amplified simultaneously, and the sequencing library is constructed with the pre-amplified products (see Methods for details). After the co-amplified library is sequenced, data processing is required to filter and separate DNA/RNA reads. After the reads separation, additional adaptor sequences are removed. Following reads preprocessing, the DNA and RNA data can be analyzed separately using standard single-cell analysis computational pipelines.

### Example 2—Comparison of Subject Methods to SmartSeq2

To benchmark the method, we used HCT116 colon cancer cell line to compare the single cell RNA data generated by this method with data generated by the current standard for single-cell RNA-Seq, called SmartSeq2. SmartSeq2 is used to profile RNA only from a single cell. Due to the difference in chemistry, we expect a dramatic difference between data generated by our method that uses a total RNA capture, compared to SmartSeq2 that employs polyT selection process. Our method is very comparable in performance metrics such as gene detection sensitivity, read coverage across the genome, and gene body coverage for transcripts (FIGS. 1B-1C; FIG. 6F). We also showed by merging groups of single cells together to reconstruct a “pseudobulk”, that the DNA copy number changes measured by our method are comparable to using bulk DNA sequencing (FIG. 1I-K).

To benchmark the method against other single-cell DNA/RNA co-profiling methods, we took published data from previously developed methods DR-seq²³ and G&T-seq (see Macaulay, I., Haerty, W., Kumar, P. et al. G&T-seq: parallel sequencing of single-cell genomes and transcriptomes. Nat Methods 12, 519-522 (2015).) and compared them against our data. The DNA reads generated by our method for single-cell whole genome sequencing shows substantial improvement in sequence coverage uniformity as compared to the other methods (FIGS. 6A-6B), which is an important metric for whole genome sequencing and copy number change detection. The RNA reads generated by our method for single-cell RNA-sequencing also shows improvement in sensitivity and accuracy compared to these two other methods when we evaluated the performance using the ERCC technical standard (see Wu, A., Neff, N., Kalisky, T. et al. Quantitative assessment of single-cell RNA-sequencing methods. Nat Methods 11, 41-46 (2014). And The External RNA Controls Consortium. The External RNA Controls Consortium: a progress report. Nat Methods 2, 731-734 (2005). https://doi.org/10.1038/nmeth1005-731.) developed by the National Institute of Standards and Technology, as we could detect a higher number of ERCC RNA transcript species in a given single cell and we could detect them more accurately over many cells, even at low concentrations of each RNA molecule (FIG. 6C).

### Example 3—Co-Amplifying RNA and DNA in a Human Tissue Sample

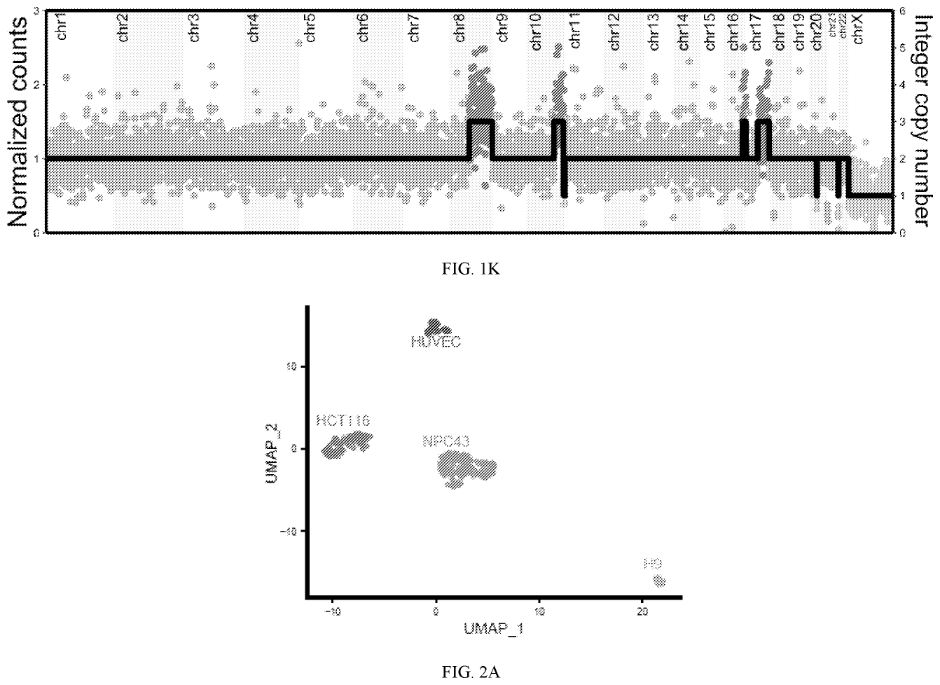

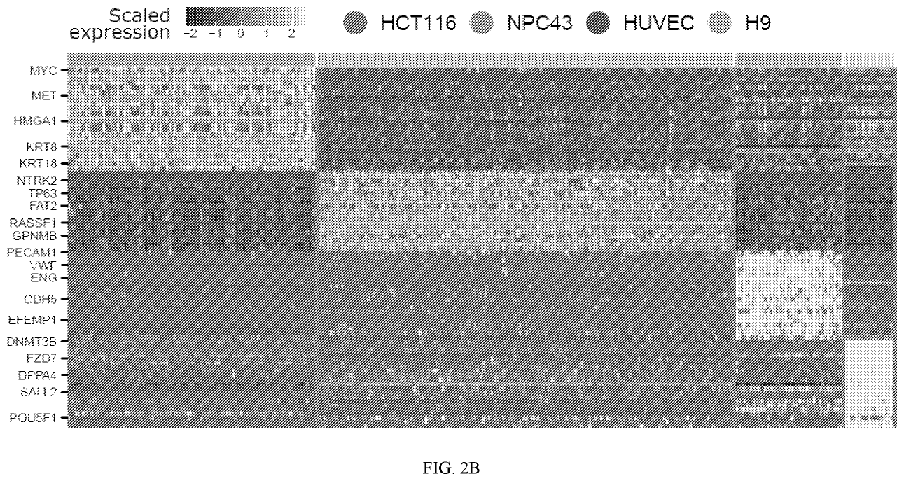

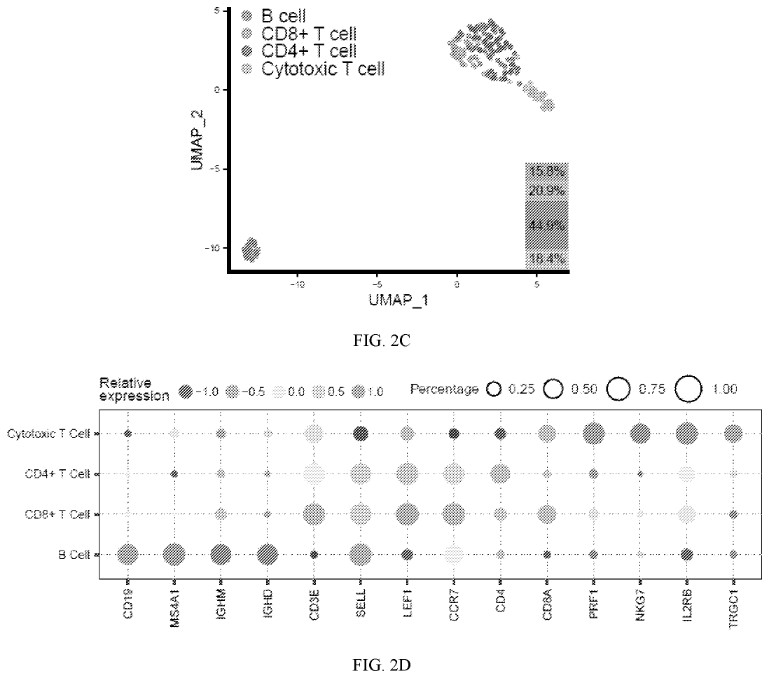

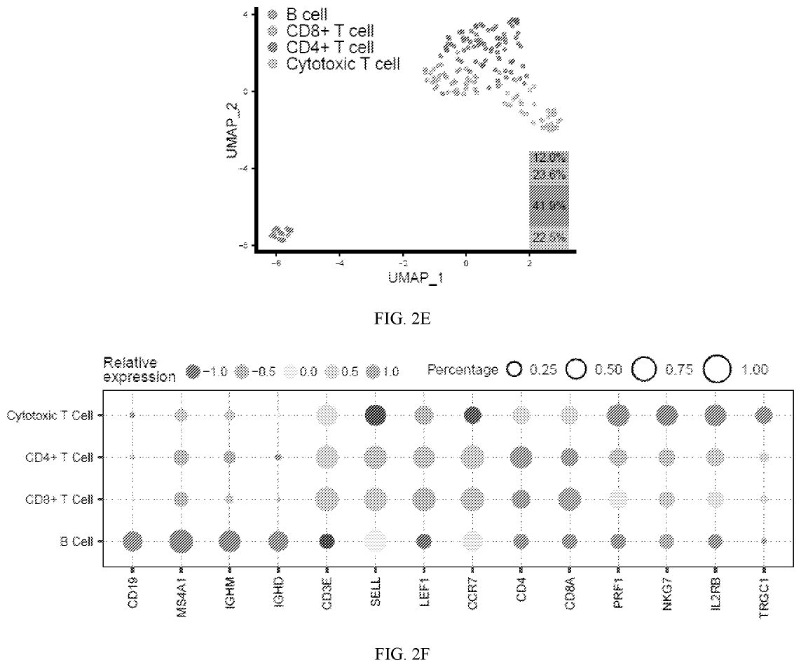

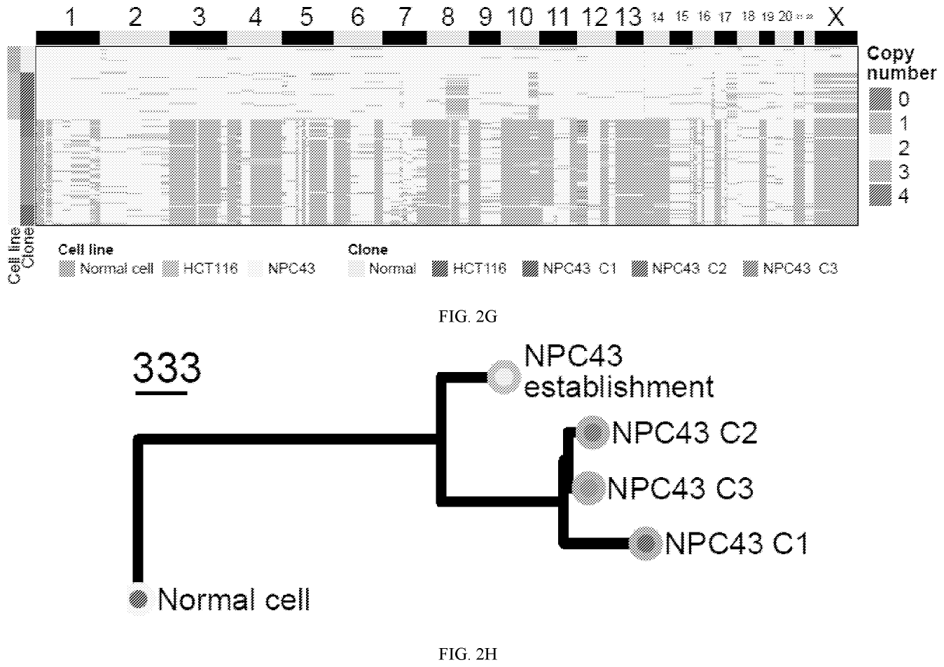

We next demonstrated our method on primary human cells to show that it can be used on fresh tissue samples. We successfully co-profiled DNA and RNA from a sample of peripheral blood mononuclear cells (PBMCs) isolated from human whole blood using our method. From this data it is possible to identify all the expected cell populations such as B-cells, T-cells and NK cells, based on the gene expression markers (FIGS. 2C-2F; 7A-7S). The DNA data showed no copy number changes, as this was blood from a healthy donor. We performed a similar analysis on four different cell lines to demonstrate that different genome and transcriptome profiles can be distinguished using data from our co-profiling method. Like the PMBCs, we are able to use the RNA portion of the data generated using our method to cluster cells from each of the different cell lines correctly, and show the relevant gene expression markers (FIG. 2A-2B). Unlike the PBMCs, two of the four cell lines we chose are cancer cell lines with copy number alterations (CNAs), and we were able to observe the CNAs in the DNA portion of the data generated using our method. As expected, we did not observe CNAs in the non-cancer cell lines (FIG. 2G).

### Example 4—Co-Amplifying RNA and DNA of Epstein-Barr Virus in Nasopharyngeal Carcinoma Samples

We also showed the applicability of our method to probe virus-host interactions using nasopharyngeal carcinoma (NPC) as an example. NPC is a type of cancer that harbors Epstein-Barr virus (EBV), and due to the viral interactions this cell type harbors both transcriptomic and genomic heterogeneity. By co-profiling DNA and RNA from an EBV+

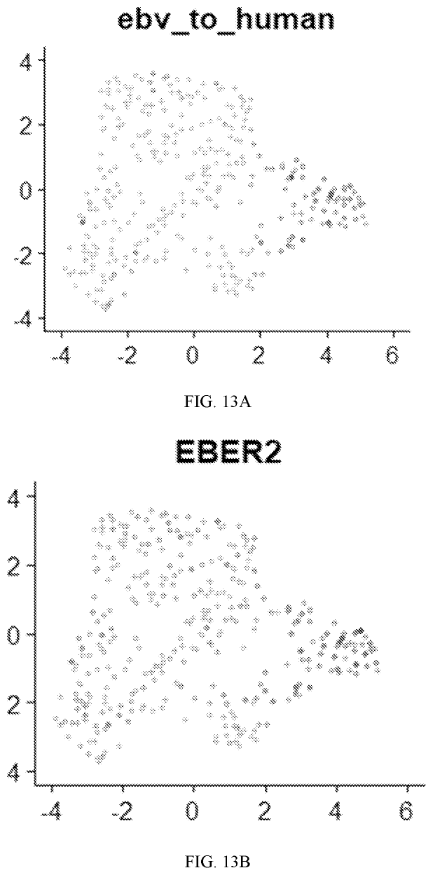

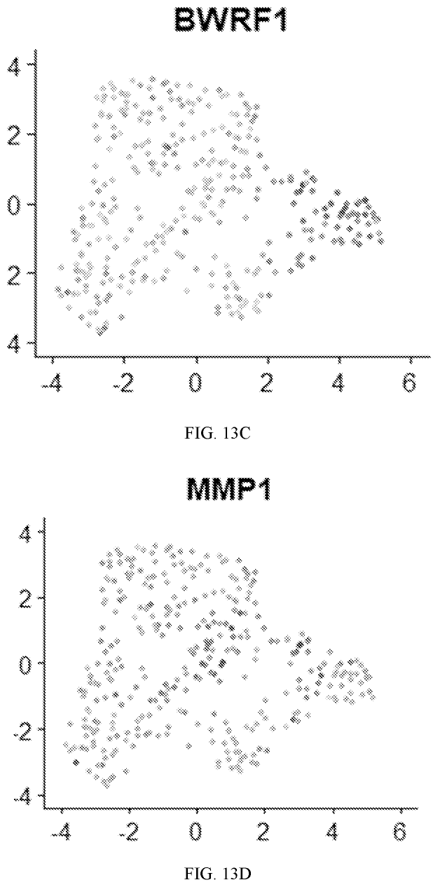

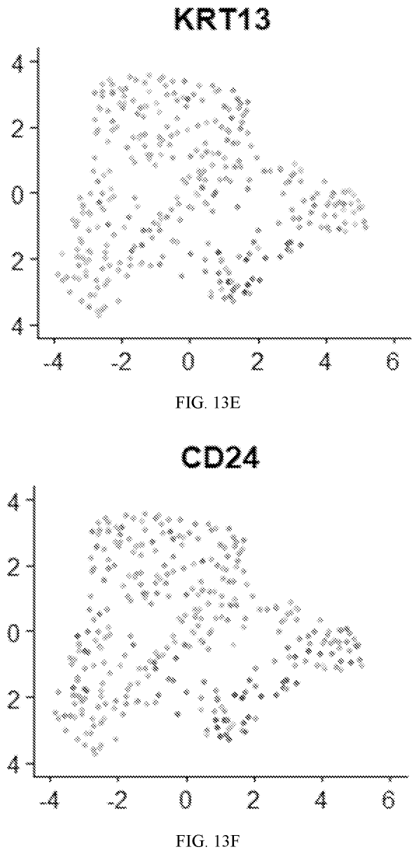

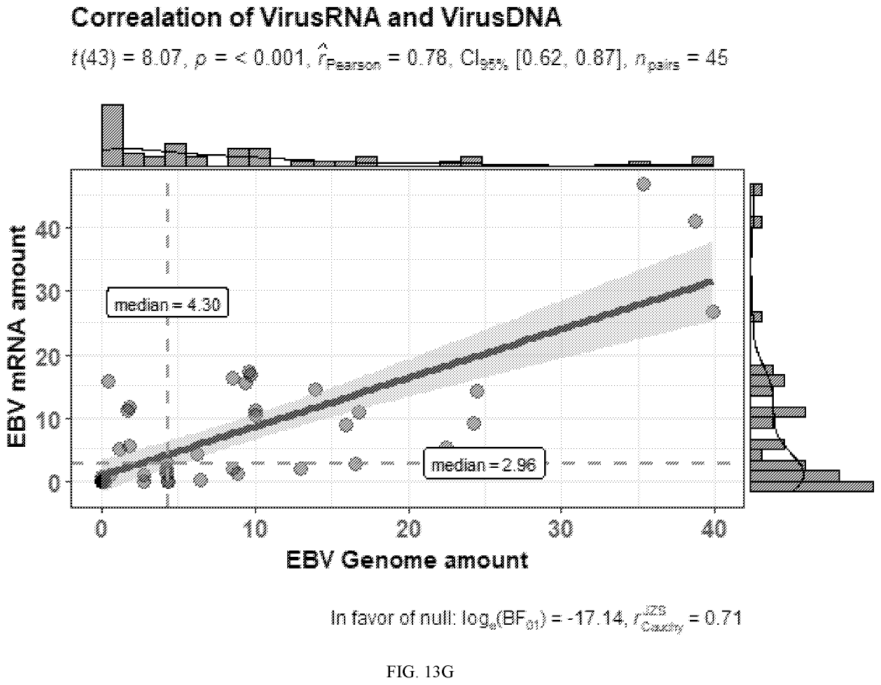

NPC cell line using our method, we were able to observe the heterogeneous virus distribution among NPC cancer cells (FIGS. 13A-13F). And the virus abundance within the host cell showed a strong correlation with the virus gene expression (FIG. 13G). Using the virus abundance information, we correlate all genes with the virus abundance and selected top correlated genes for downstream virus active pattern analyze. With these genes, cells could be separated into virus-rich cells and virus-poor cells.

### Example 5—Molecular Barcoding Strategy Enables Accurate and Sensitive Co-Profiling of DNA and RNA from a Single Cell in a One-Tube Reaction

To characterize the transcriptome generated by scONE-seq, we benchmarked it against Smart-seq2 (SS2)^{36,37} using a variety of test samples: extracted RNA-free E. coli genomes (mock DNA), extracted DNA-free human total RNA (mock RNA), as well as a mixture of the two (i.e., E. coli DNA mixed with human total RNA); and cultured HCT116 single cells. We evaluated the sensitivity by assessing the number of genes detected in each of the benchmark mock and HCT116 samples and found that scONE-seq detected more genes per cell than SS2 (FIG. 1B and FIG. 6D; p<2×10⁻¹⁶; t-test). This is likely due to scONE-seq being able to capture total RNA^{34,38} while SS2 only targets polyA RNA, and therefore capturing a more diverse set of molecules at any given sequencing depth (Supplementary FIG. 1d). Also, scONE-seq enables full-length transcript profiling and achieves gene body coverage uniformity comparable to SS2 (FIG. 1C). We then used sample-to-sample correlation analysis as well as the detection of ERCC spike-ins to estimate the accuracy of scONE-seq in comparison to SS2. In the sample-to-sample correlation analysis, the coefficient of determination (R2) is comparable for the two methods (FIGS. 1D-1G; FIG. 6C). Using ERCCs as a measure of accuracy, scONE-seq and SS2 are comparable in performance, meaning the accuracy of scONE-seq is sufficiently high for quantitative measurement of transcript abundance from single cells (FIG. 6C).

Next, we sought to validate the whole genome sequencing (WGS) capability of scONE-seq. Lorenz curves³⁹ compare the coverage uniformity for each method, showing a good performance by scONE-seq (FIG. 111; FIG. 6A-6B). Then, we used bulk HCT116 WGS data in comparison to scWGS data generated by scONE-seq to confirm that CNAs captured by scONE-seq are consistent with those defined by bulk (5×10⁶ cells) and pseudo bulk (86 cells; 500-kb resolution) (FIGS. 1I-1K). In addition, we performed UMI deduplication on the scONE-seq DNA dataset, since our method adds UMI to the DNA fragments during the tagmentation step, and found that this deduplication successfully reduces the bias introduced during single-cell DNA amplification.

Summarily, the analysis of scRNA-seq and scWGS data generated using benchmark samples shows that scONE-seq can profile genome and transcriptome data from the same single cell without compromising data quality as compared to existing standard methods.

### Example 6—scONE-Seq Data Correctly Assigns Cell Types from Primary Donor Samples

After thoroughly assessing the technical performance of scONE-seq, we next applied it to known biologically heterogeneous samples to evaluate whether it can accurately identify cellular subtypes within a mixed population. To do so, we performed scONE-seq on four different cell lines, as well as on a primary peripheral blood mononuclear cell (PMBC) sample from a healthy donor.

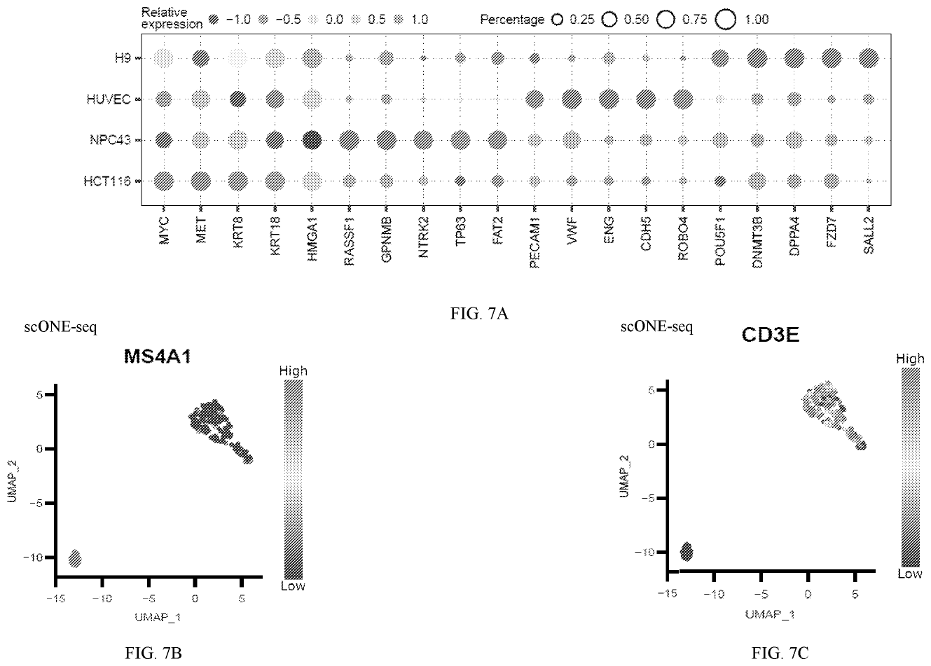

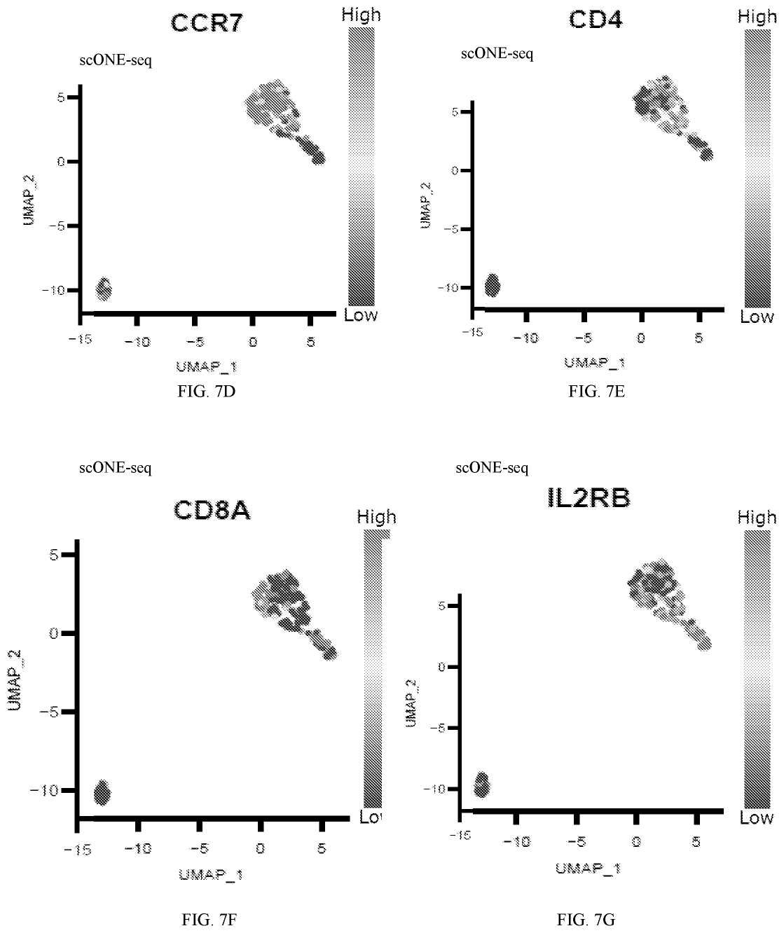

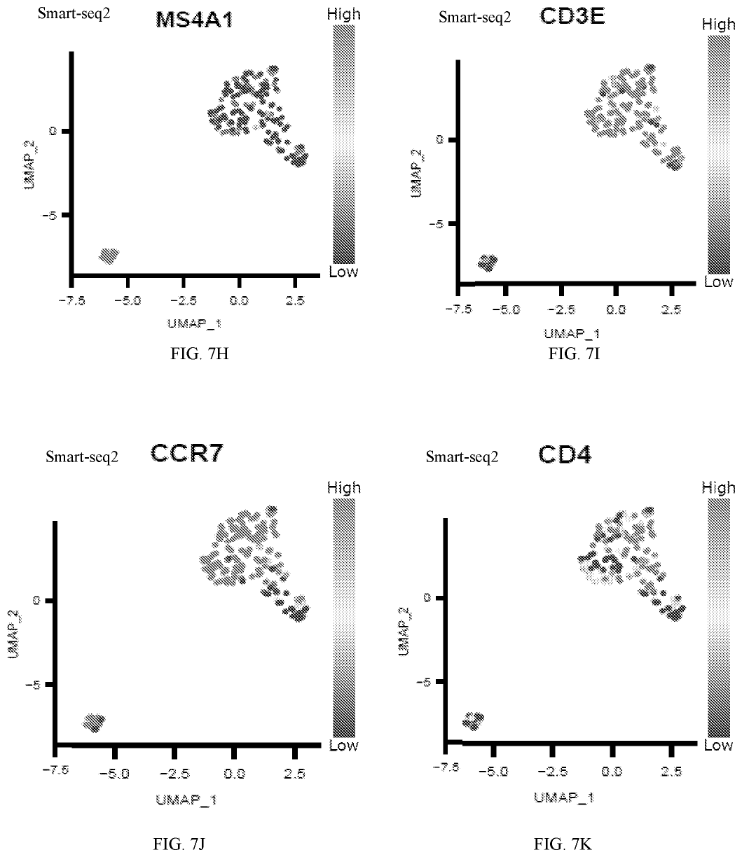

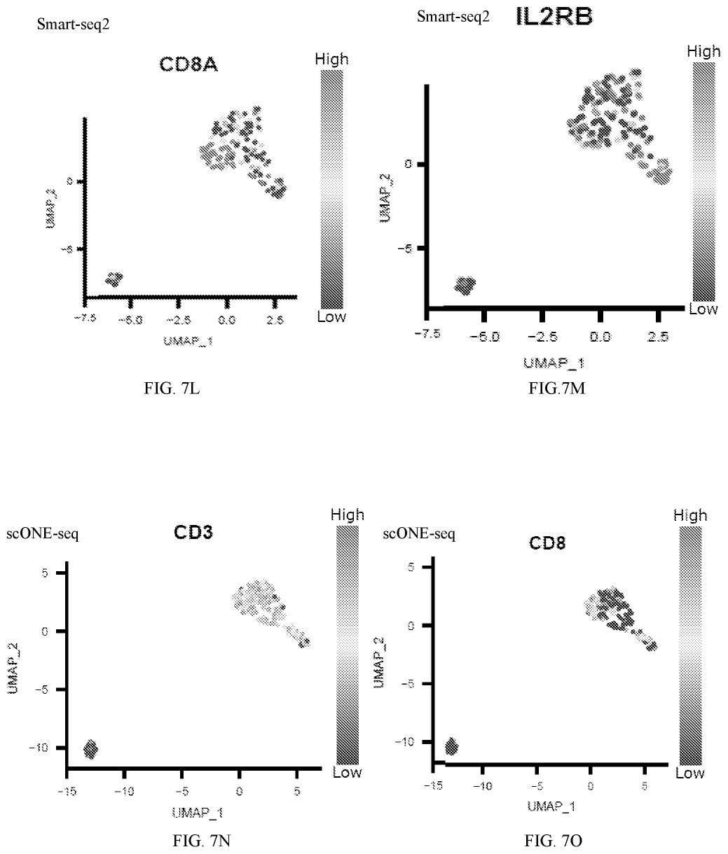

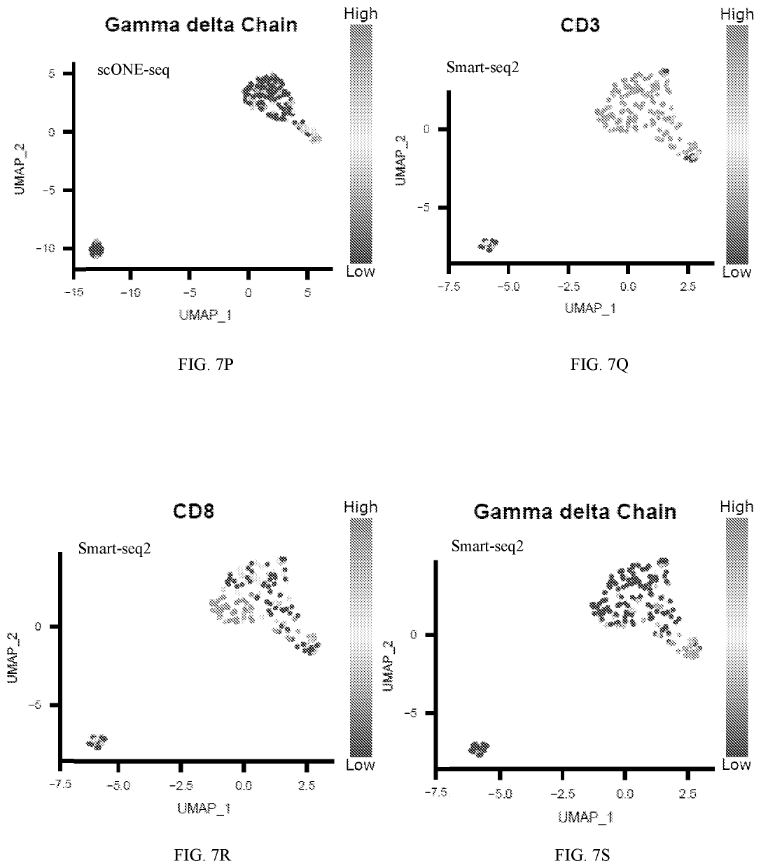

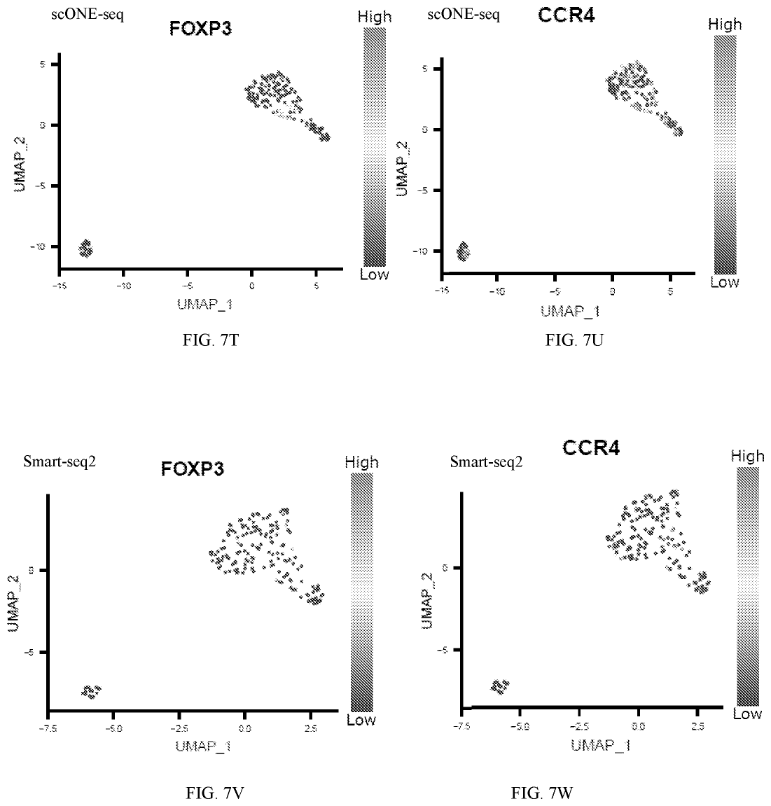

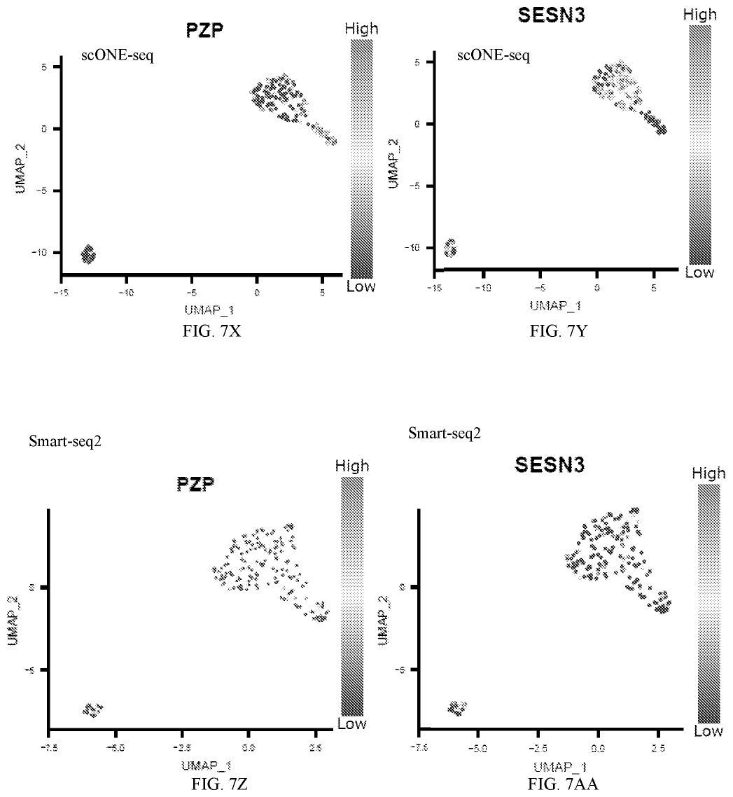

First, we analyzed the cell-line dataset containing 86 HCT116 cells, 143 NPC43 cells, 37 HUVEC cells, and 17 H9 cells to check for accurate cell-type assignment. With unsupervised graph-based clustering, cells from the same cell lines successfully clustered together (FIG. 2A). We also checked the gene markers for each cell line (FIG. 2B) and notably, several well-studied gene markers for these cell lines are found in the scONE-seq dataset (FIG. 7A).

Next, we used lymphocytes from PBMC to test scONE-seq cell-types clustering accuracy in primary samples. We prepared sequencing libraries with scONE-seq and SS2 from the same PBMC sample for comparison. After quality control filtering to remove low-quality cells and potential doublets (see Methods), we collected 200 cells for scONE-seq and 194 cells for Smartseq2. With unsupervised graph-based clustering, we found no difference in the cell-type composition between the two methods (FIG. 2C and FIG. 2E; p=0.1826; Chi-squared test). After clustering, we annotated the cell types using known lymphocyte markers⁴⁰⁻⁴² (FIG. 2D and FIG. 2F, and FIGS. 7B-7S): B cells distinguished by CD19 and MS4A1 (CD20); T cells characterized by CD3E; Less differentiated T cells (Naïve and memory T cells) identified by SELL CD62L, CCR7, and LEF1; CD4+T cells characterized by CD4; CD8+ T cells characterized by CD8A and CD8B; and cytotoxic T cells distinguished by PRF1 and NKG7. Notably, cytotoxic T cells comprise both Gamma delta T cells (γδ T cell; expressing TRDC, TRGC1, and TRGC2; FIGS. 7P-7S) as well as effector memory T cells (TEM; lacking CCR7 expression and positively expressing IL2RB). We surmised that these are TEM rather than effector T cells since the sample is from a healthy donor (FIG. 7B-7M). Beyond that, in the scONE-seq dataset, we also captured some regulatory T cells (Treg cell; FOXP3+, CCR4+) (FIGS. 7T-7W) and detected several non-polyA genes including PZP and SESN3 whose expressions in T-cells have previously been described⁴³ (FIGS. 7X-7AA); these features were not found in the SS2 dataset.

These results collectively demonstrate that scONE-seq RNA data can accurately capture the biological variation within a heterogeneous sample.

### Example 7—scONE-Seq Data Identifies Distinct Clones in Different Samples

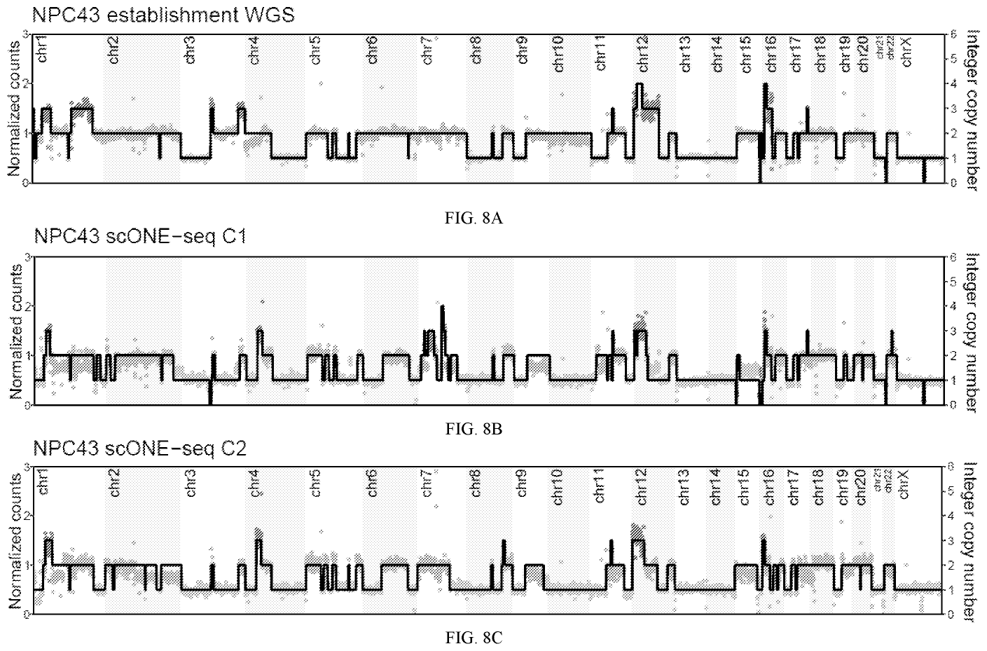

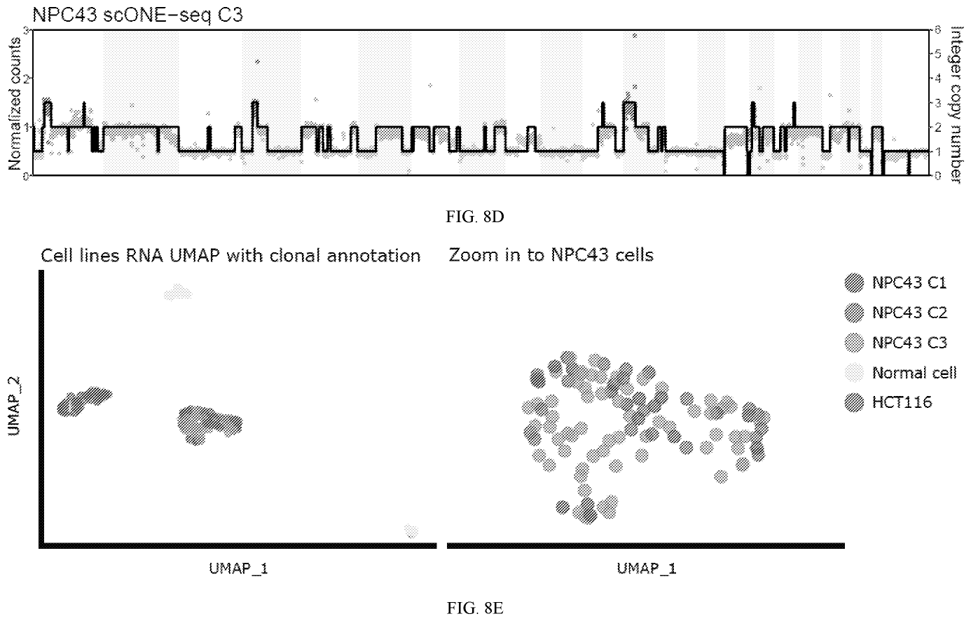

The analysis above shows the feasibility of scONE-seq for the cell-type assignment using RNA data. Next, we evaluated the performance of clone identification with scONE-seq WGS data. Here, we utilized scONE-seq WGS data that was obtained simultaneously from the cell lines used in the previous cell-type assignments analysis, and delineated the CNAs clonal structure of all four cell lines, followed by hierarchical clustering with their copy number profiles (FIG. 2G). From this analysis, we see that HCT116 maintains a relatively homogeneous clonal composition, whereas NPC43, a primary patient-derived cell line that shows strong genome instability, is comprised of 3 main clones (FIG. 211). Furthermore, the CNAs structure of these 3 clones differ substantially compared to when the cell line was first established⁴⁴ (FIGS. 8A-8D), especially in chromosomes 1, 3, 4, 6, and 7. Correspondingly, distinctions between clones are mainly found in chromosomes 1, 3, 7, and 11 (FIGS. 8A-8D). Based on this observation, the change in chromosome copy numbers during cell culturing of primary cell lines could be a common phenomenon in cell lines with abundant CNAs and unstable genomes. Studies have shown extensive genetic variation across different cell culture lines, and that single cells from some cell lines can give rise to populations with multiple clones due to genome instability^{20,45}. Additionally, with the matched transcriptomes of each single cell and their corresponding copy number states, we mapped the clonal information to the transcriptome UMAP for NPC43 and found that the CNAs in NPC43 did not impact the transcriptome state dramatically (FIG. 8E). This demonstrates that scONE-seq can identify both phenotype and genotype states for each individual cell.

### Example 8—Dissecting the Clonal Structure and Cell-Type Subpopulations of an IDH-Mutant Glioblastoma

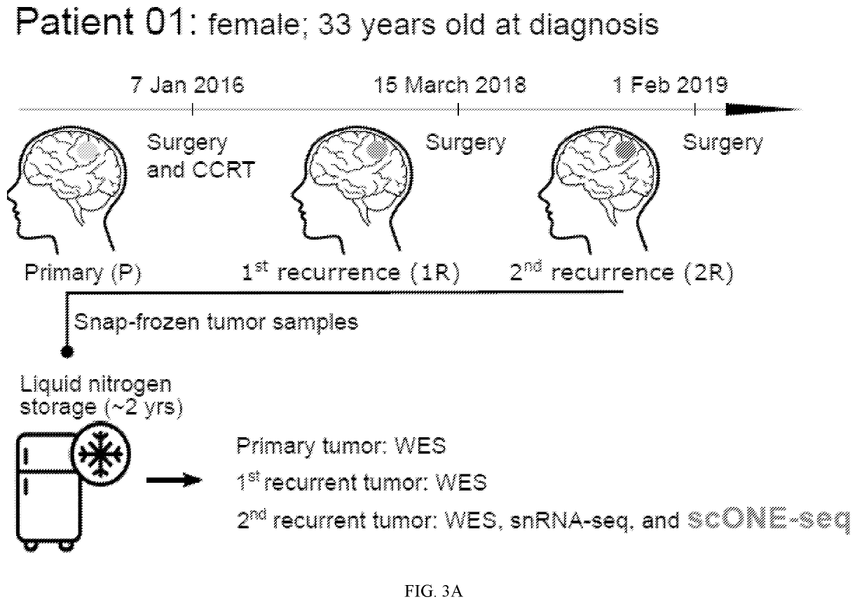

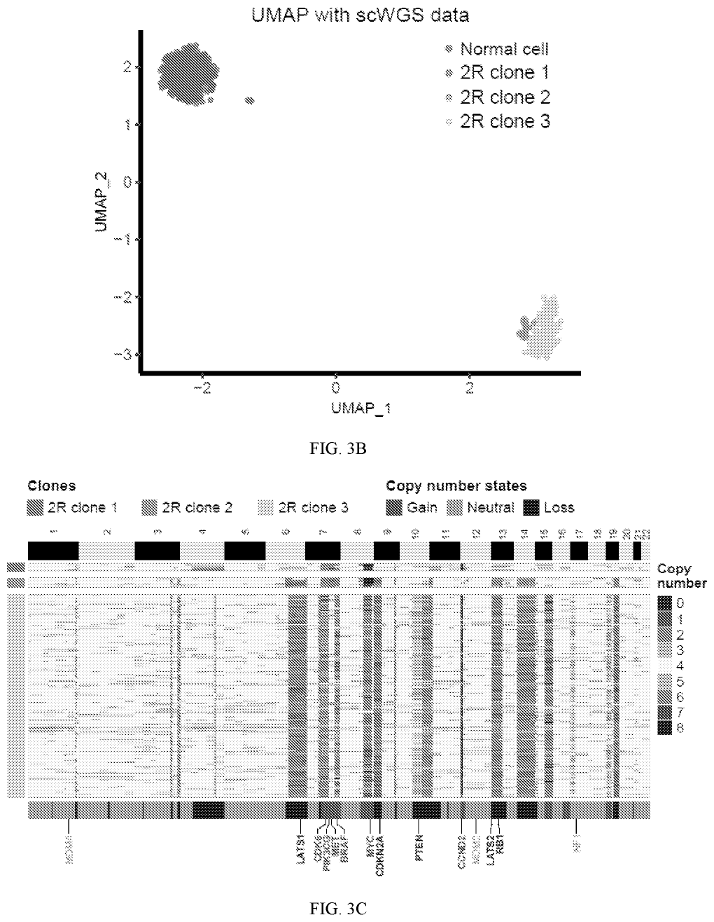

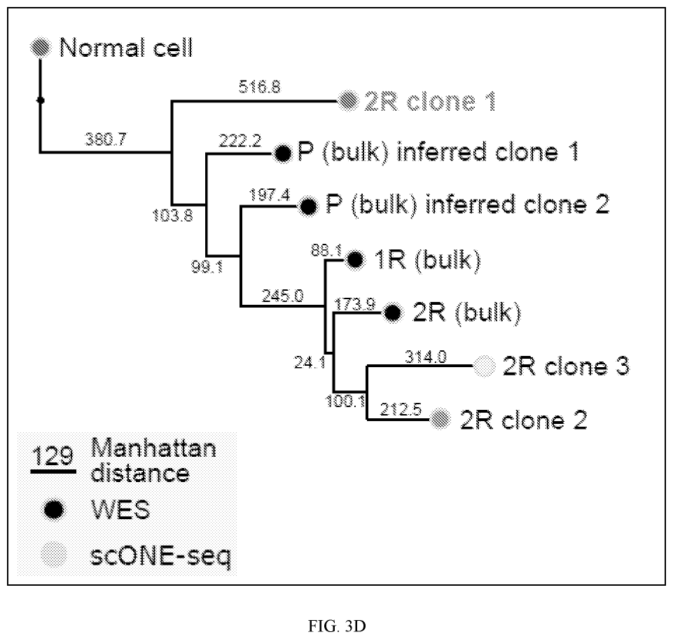

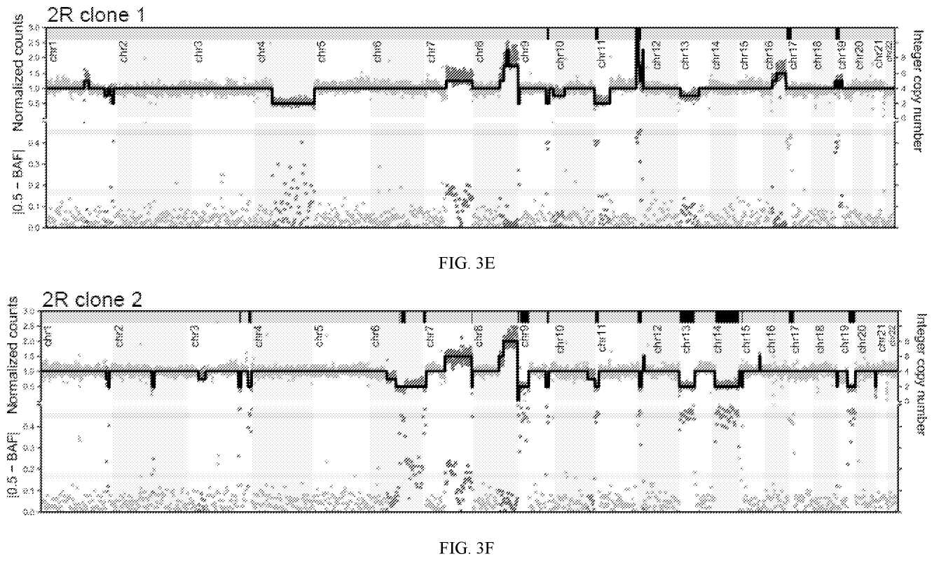

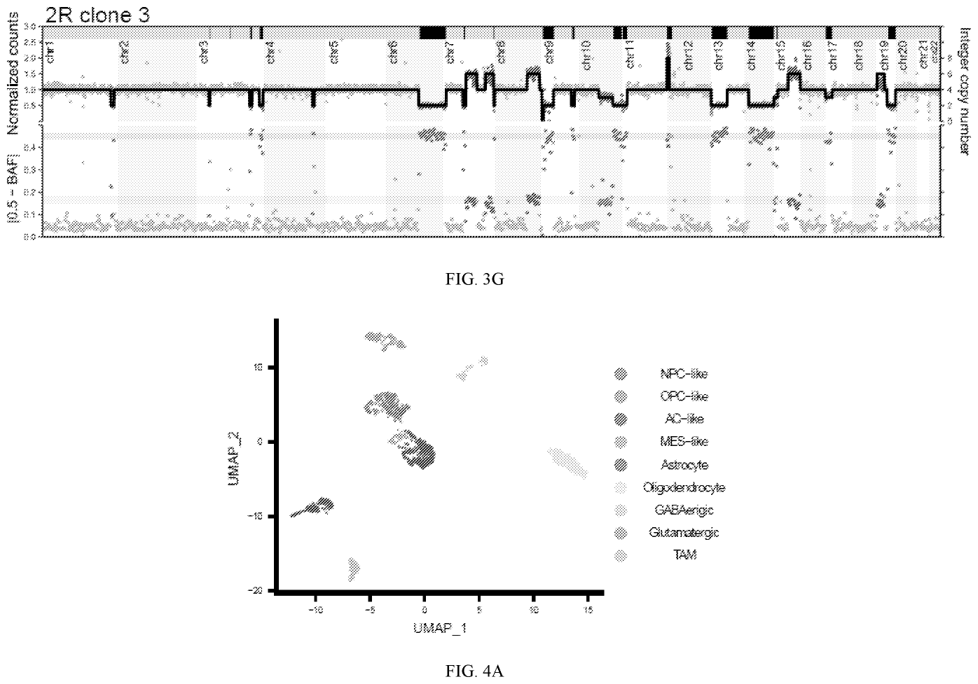

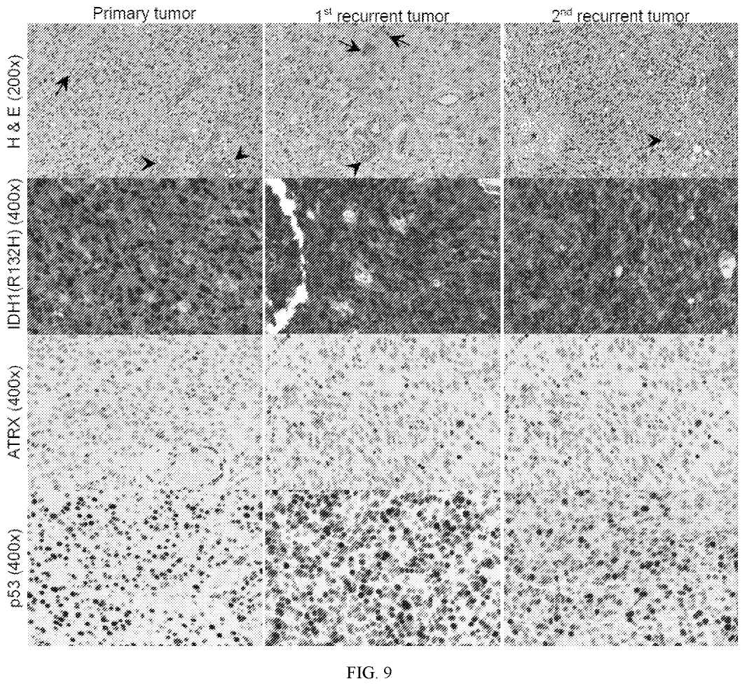

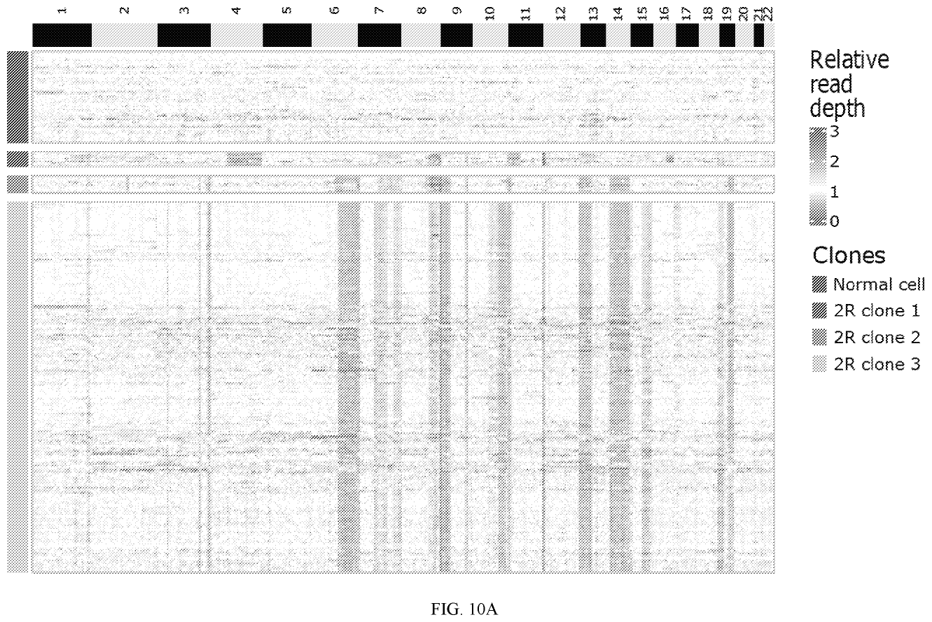

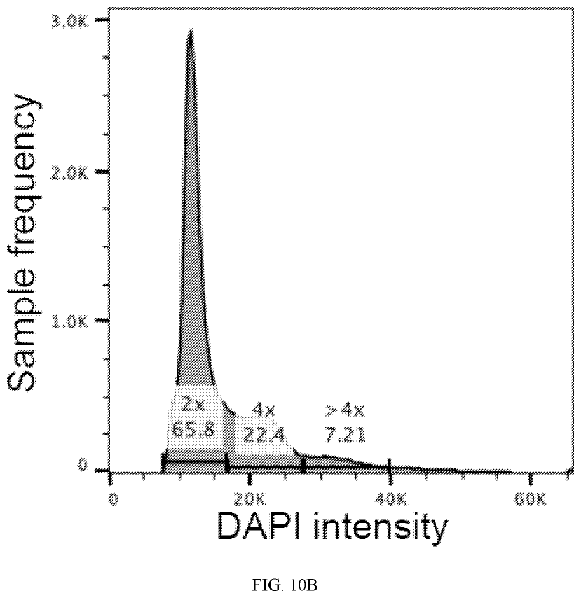

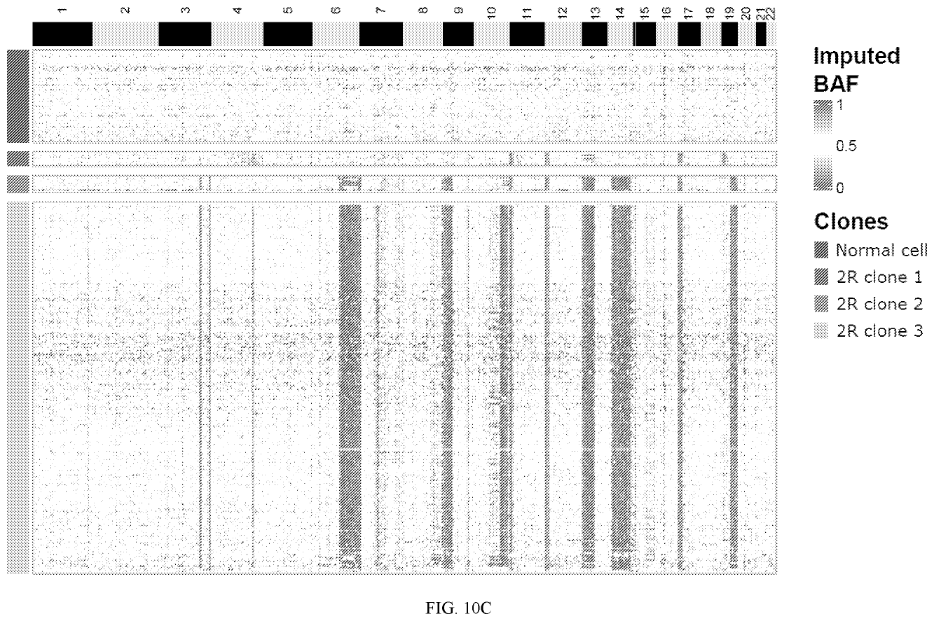

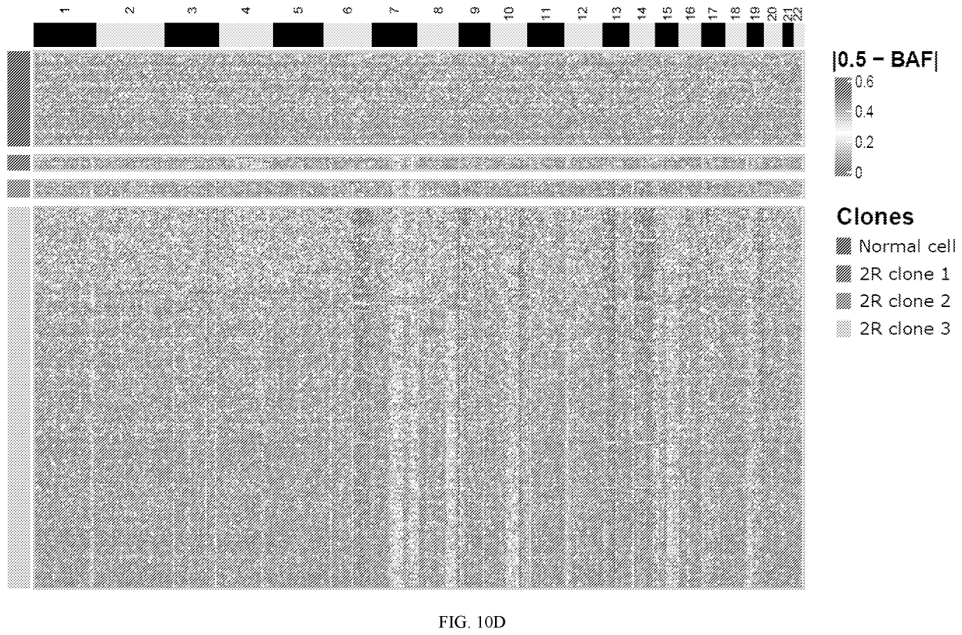

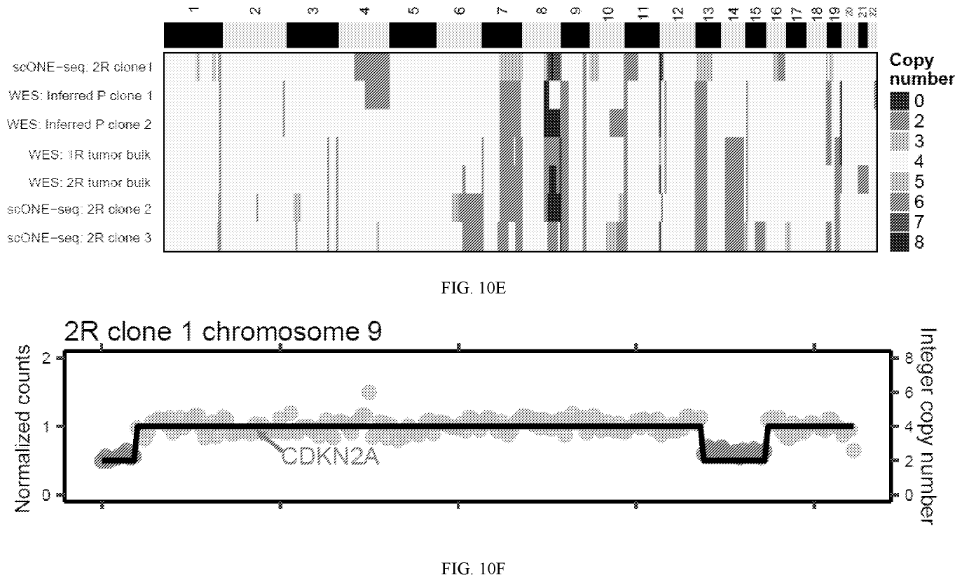

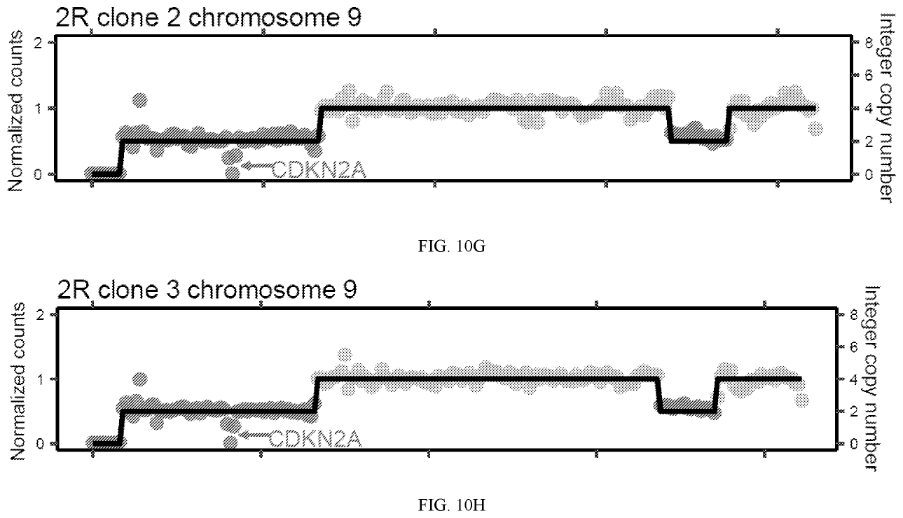

Glioblastoma (GBM) is one of the most aggressive malignant tumors originating in the brain^{46,47}. When studying GBM or other brain tissues using single-cell technology, it is challenging to obtain intact dissociated whole single cells, especially neurons with their complex morphology, and could lead to biases in cell-type sampling⁴⁸. As such, for brain single-cell profiling, single nucleus isolation is more widely used. To profile both the genotypic and phenotypic heterogeneity in a biobanked GBM sample, we apply scONE-seq on single nuclei isolated from a months-old snap frozen GBM specimen: a second recurrent GBM sample with IDH1 (R132H), TP53 (P278S), ATRX (R781*) mutation (FIG. 3A; FIG. 9). The primary and first recurrent samples were limited in quantity, and subject to whole exome sequencing (WES) and RNA sequencing in bulk (FIG. 10E) In total, we used scONE-seq to profile over 1200 nuclei, including 1210 scRNA datasets, 1089 scWGS datasets, generating 908 passed-QC paired DNA and RNA datasets.

First, we delineated the clonal structure of this GBM sample. Using dimension reduction with normalized counts data (500 kb genome bins) we clustered cells into four distinct genomic states (FIG. 6A); hierarchical clustering was used to identify these four groups of cells as one cluster of normal cells and three GBM clones (FIG. 10A). Meanwhile, genome-wide duplication was found in this tumor and validated by measuring each cell's DAPI intensity using flow cytometry, as well as using the B-allele frequency, which revealed aneuploidy and loss of heterozygosity in multiple loci (FIGS. 10B-10D). Considering the aneuploidy, the integer copy number of cells was calculated (FIG. 3C). Thereafter, the clonal pseudo-bulks were used to present the existence of the loss of heterozygosity (LOH) (chr6q, chr9p, chr10q25.1-chr10q26.2, etc.) and imbalanced allele frequency (chr10q21.2-chr10q24.33, etc.) (the chr10q21.2-chr10q24.33 region contain at least 3 copies), suggesting that whole-genome duplication (WGD) events had occurred (FIG. 3C). Based on the genomic profile of each clone and the calculated Manhattan distances between them, clone 1 was found to be closer to the root (normal cell), with fewer loss of heterozygosity (LOH) events, and it has similar genome alterations as the primary tumor WES data (FIGS. 3C-3G). Clone 2 and clone 3 harbor many of the same deletion regions as clone 1 and the primary tumor, resulting in LOH (FIGS. 3E-3G). Notably, the chr6 deletion in clone 2 and clone 3 are allele-specific (FIG. 10E), which indicates that this deletion occurred after other LOH events. In addition, we surveyed the commonly altered GBM and IDH-mutant glioma driver genes^{49,50} and found BRAF, MET, and MYC are amplified in all clones (FIG. 3C). In contrast, deletion events are quite different from the amplification events, and many of them only occur in clone 2 and clone 3, including deletion of CDKN2A, and PTEN. Importantly, the homo-deletion of CDKN2A was found (FIGS. 10F-10H).

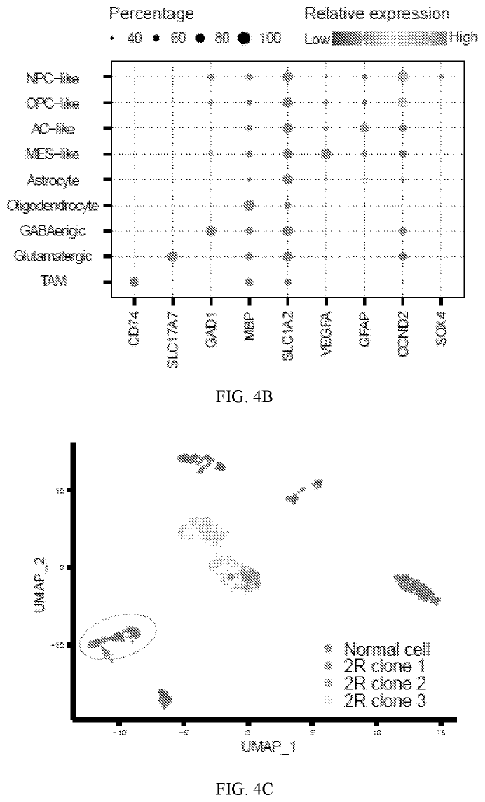

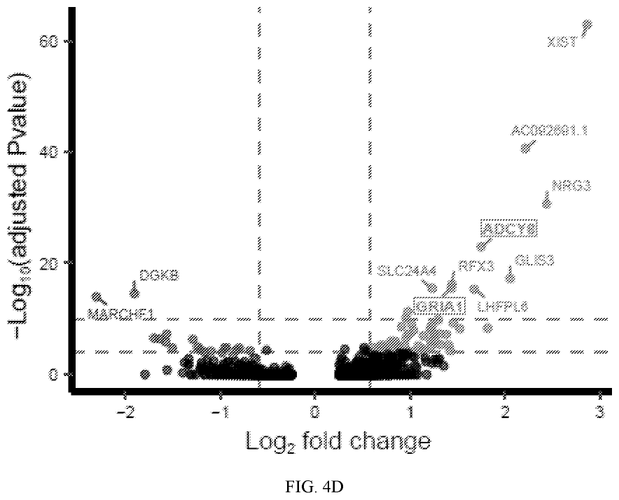

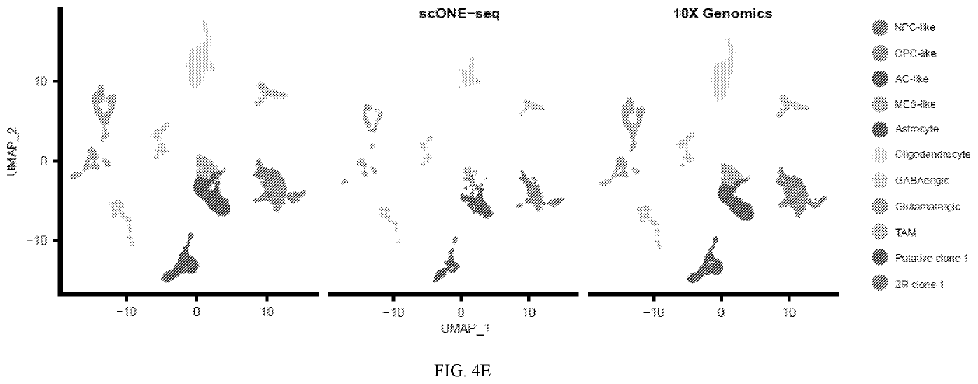

Next, we analyzed the RNA data from this dataset. First, we performed unsupervised graph-based clustering on scONE-seq RNA data, obtaining multiple cell clusters that were then annotated based on their RNA markers. We found this tumor contains macrophages, neurons, astrocytes, oligodendrocytes, and tumor cells based on canonical cell type gene signatures (FIGS. 4A-4B). The complex tumor microenvironment (TME) indicates a highly infiltrated tumor phenotype. The tumor cells display high EGFR expression, a well-known feature of GBM. These EGFR-high tumor cells can further be subset into 4 cellular states based on meta-modules scores described by Neftel et al.¹² (FIG. 4A): oligodendrocyte progenitor cell-like (OPC-like), neural progenitor cell-like (NPC-like), mesenchymal-like (MES-like), and astrocyte-like (AC-like).

In addition to the phylogenetic tree obtained from DNA data, which dissects the clonality, we are also able to use paired RNA data to superimpose the cell-type information onto the clonal information to identify clonal subpopulations with unique functional, phenotypic features. To do so, we mapped the clonal information to the RNA UMAP to visualize the clonal distribution among different cell types (FIG. 4C). Clone 3 is the major clone of this tumor and is differentiated into all 4 tumor phenotypes: OPC-like, NPC-like, MES-like, and AC-like cellular states. Clone 2 consists predominantly of AC-like cells. Clone 1 is the most interesting: using RNA data alone, all cells from clone 1 were clustered with normal astrocytes, indicating transcriptome similarity between clone 1 and normal astrocytes that is indistinguishable using scRNA-seq data alone; but upon superimposing matched genotype and phenotype information, this unique population of astrocyte-like tumor cells with clearly abnormal genotype as compared to the true normal cells are revealed (FIG. 4C).

The clone 1 subpopulation appears rare within the second recurrent tumor (2.06% of cells sampled with scONE-seq), and phenotypically resembles normal astrocytes.

### Example 9—Characterization of a Unique Tumor Clone with Normal Astrocyte-Like Phenotype

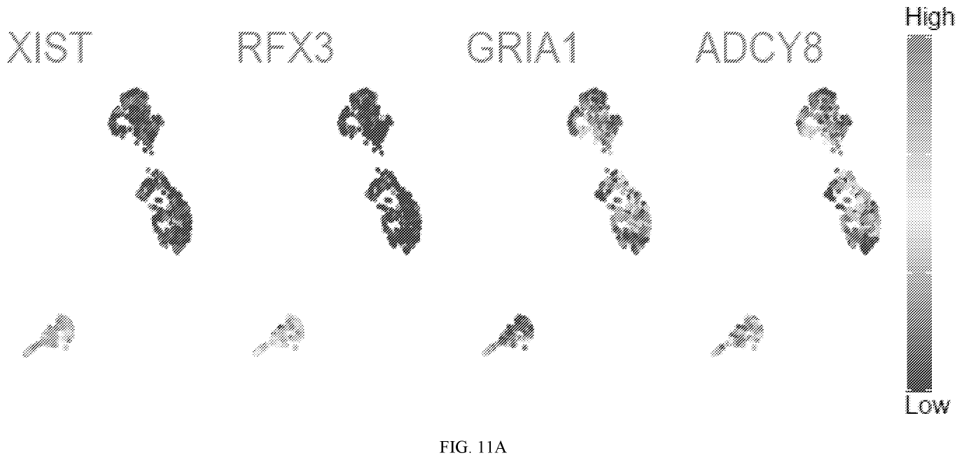

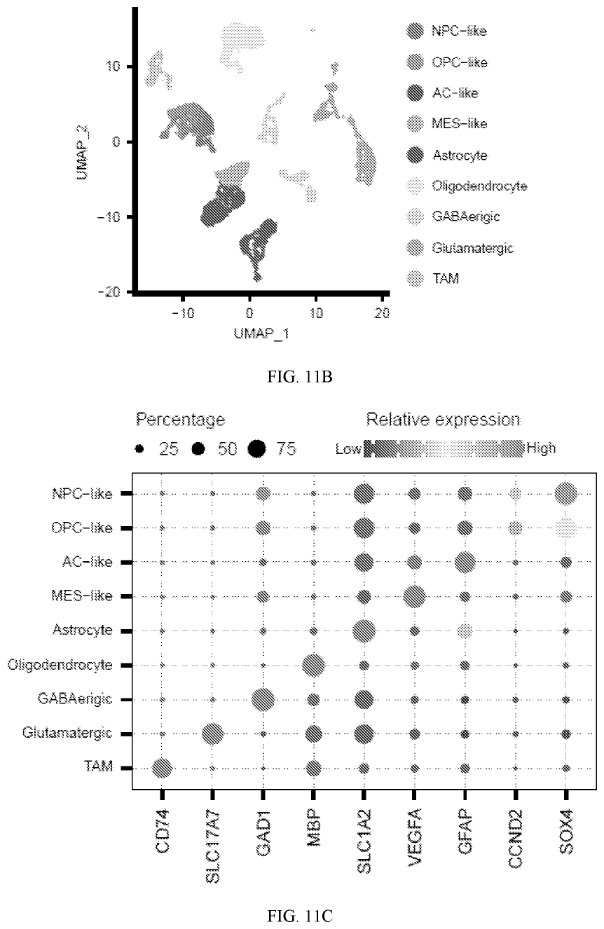

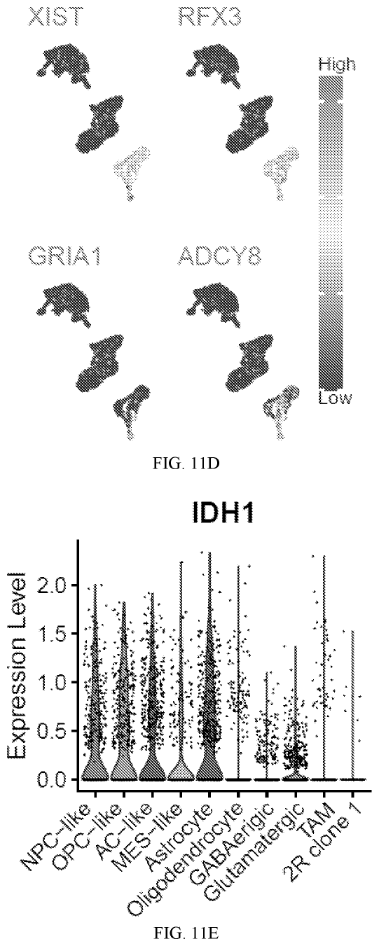

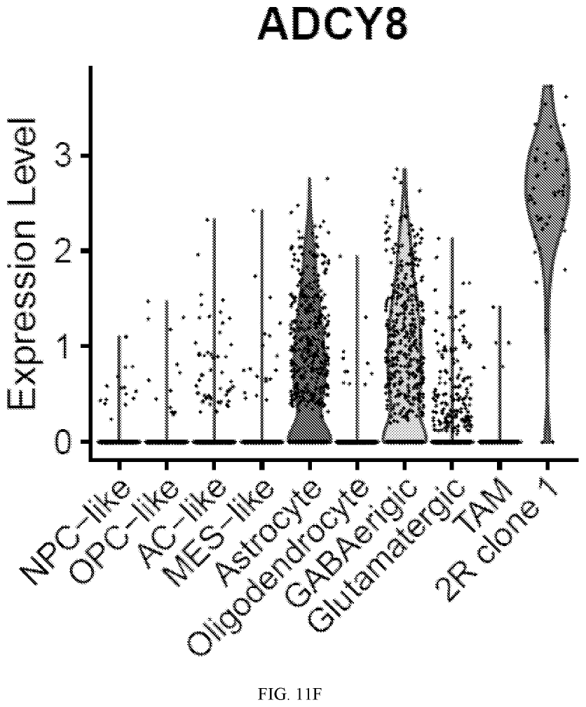

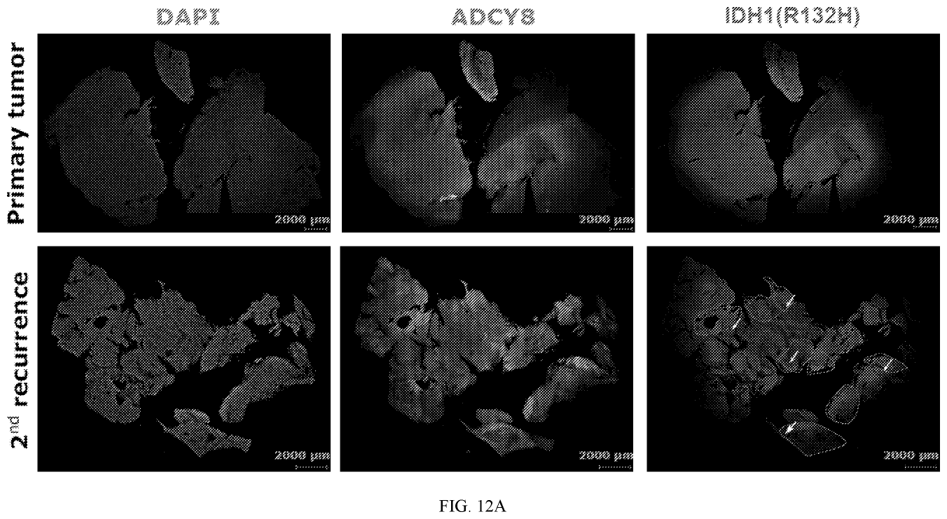

To verify the existence of clone 1 cells, we first identified gene markers unique to clone 1, including XIST, RFX3, ADCY8, and GRIA1, which can distinguish them from other subpopulations (FIG. 4D, FIG. 11A). These markers were also found in the droplet-based snRNA-seq dataset to label a putative clone 1 population, also adjacent to normal astrocytes (FIGS. 11B-11D). Then, we integrated the scONE-seq RNA dataset and 10×dataset. This integration analysis shows that our scONE-seq dataset of 1000 nuclei captured all cell types that were observed in droplet snRNA-seq of 4416 nuclei and that clone 1 clustered with the putative clone 1 cells from the 10× dataset (FIG. 4E).

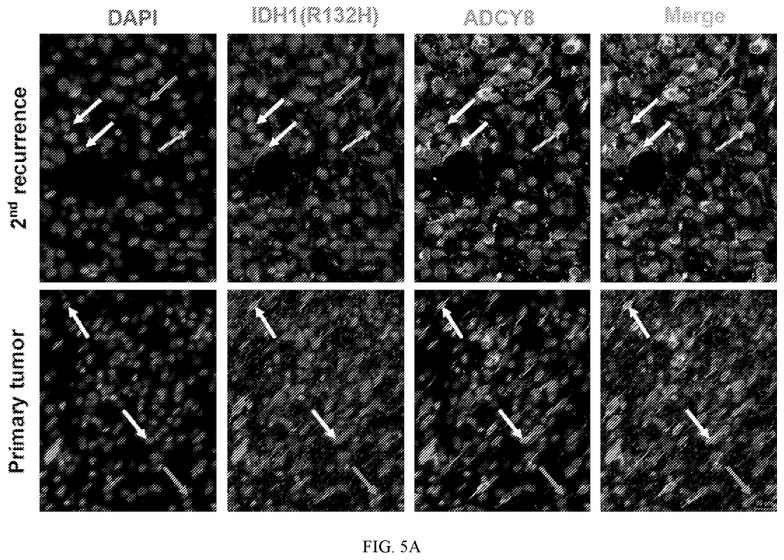

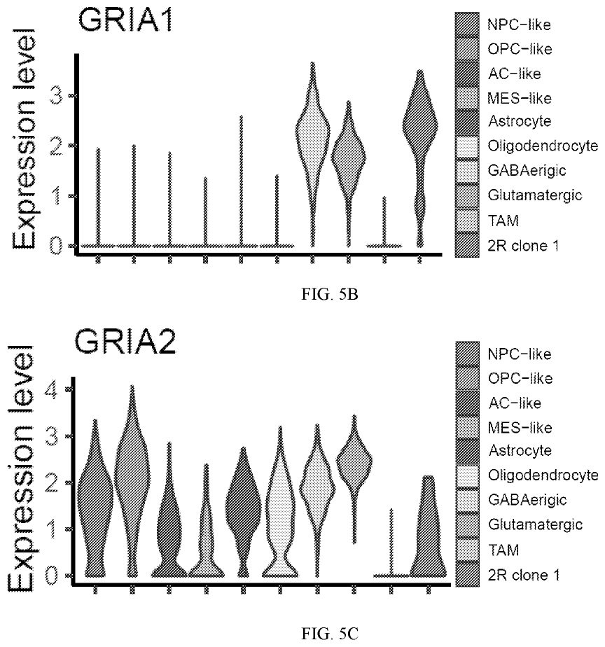

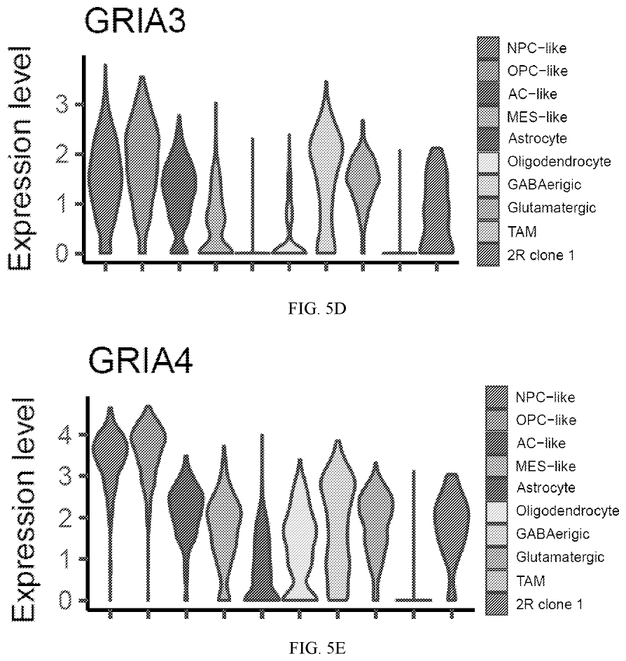

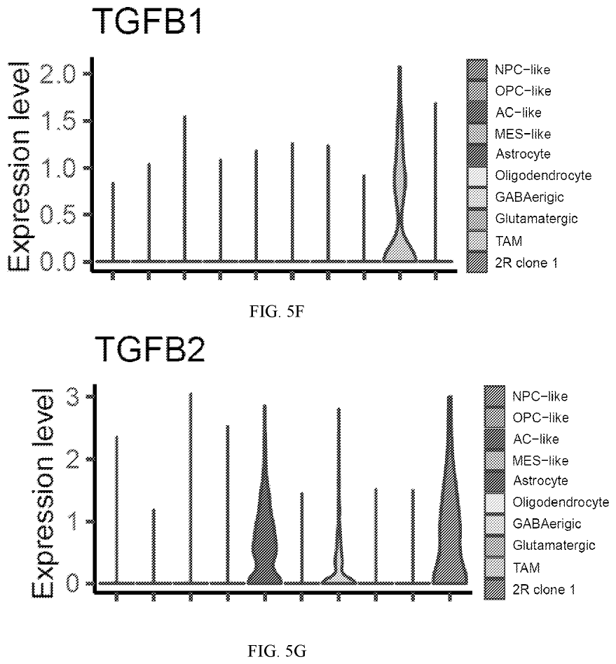

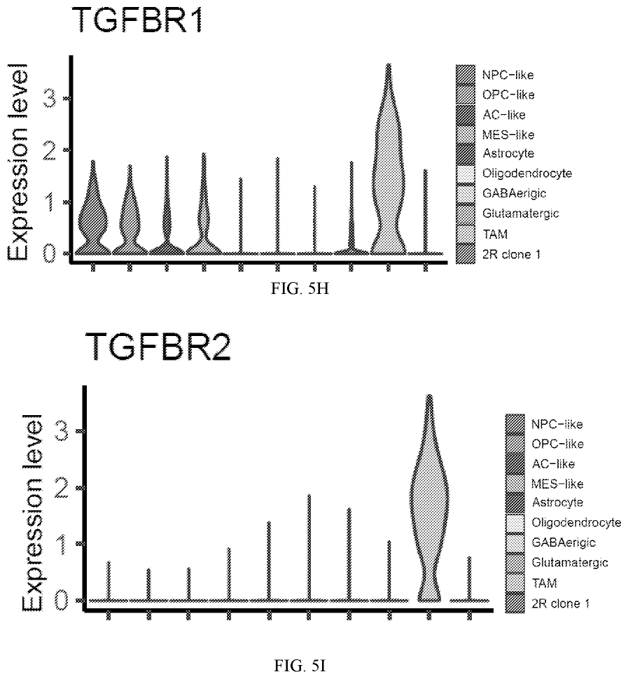

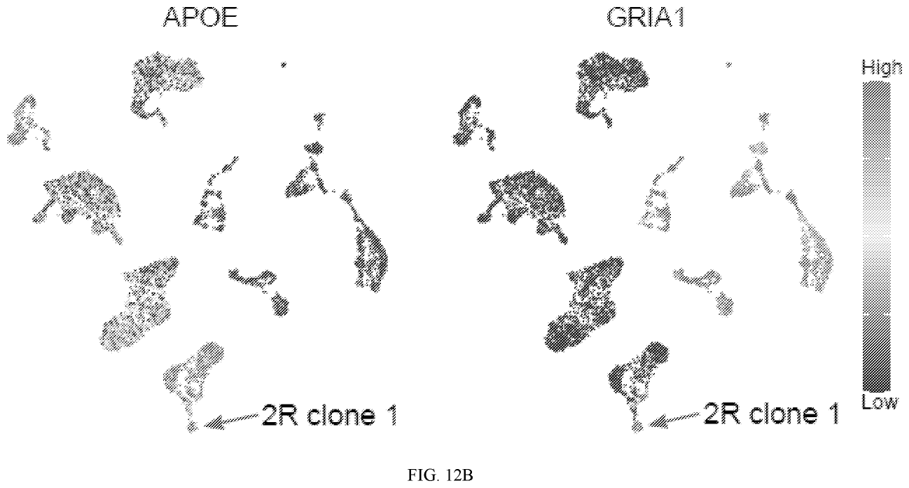

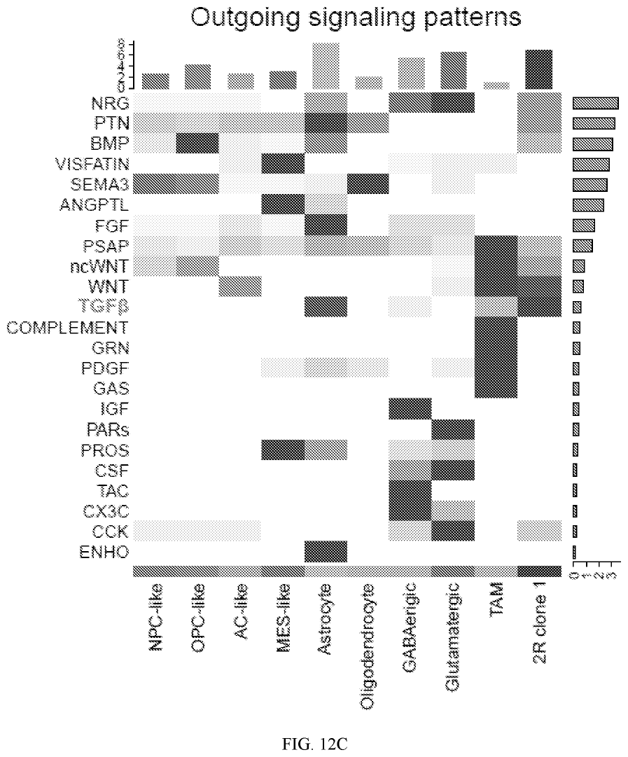

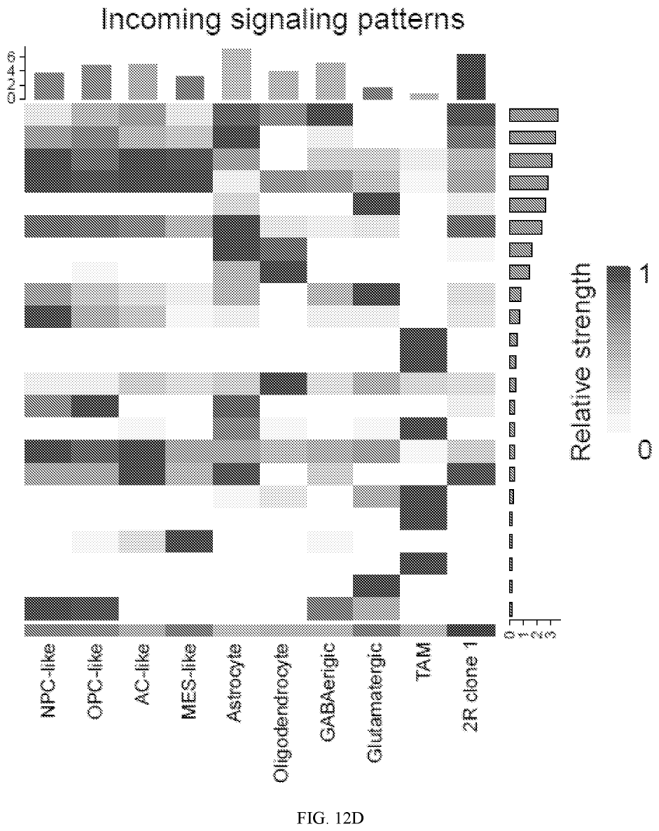

Then, we performed histological analyses on FFPE sections from the primary tumor and from the second recurrence tumor to verify the presence of clone 1 cells at different stages of tumor progression. IDH-1(R132H) was selected as the tumor marker as the patient carried IDH1 mutation, and anti-ADCY8 is expected to mark some normal neurons and normal astrocytes in addition to clone 1 cells (FIG. 4D, FIG. 11E-11F). As such, putative clone 1 cells are those cells marked by double-positive staining of IDH1 (R132H) and ADCY8. First, we looked at the overall staining pattern across the whole slide section and noted that the IDH1 (R132H) positive tumor cells are distributed over the entire section for both the primary and 2R tumors. The ADCY8 signals appear stronger in 2R tumor sections and are specifically concentrated to certain regions which also express IDH (R132H) more strongly (FIG. 12A). Interestingly, these ADCY8 positive regions are always near the IDH1 (R132H) negative ‘normal adjacent’ regions (FIG. 12A). The double-positive cells that we suspect to be the putative clone 1 cells appear to be near other normal and malignant cells (FIG. 5A). These histological immune staining results provided additional details on the spatial distribution of putative clone 1 cells in the tumor sections.

In our staining experiments, we noted that the clone 1 cells appear more abundant near the tumor margins. The presence of these tumor cells with normal-like phenotype in the infiltrated tumor regions prompted us to examine the potential role of clone 1 cells in signaling and cell-cell communication, as the infiltrated regions are an important part of the tumor microenvironment (TME). Several studies have demonstrated that glioma cells can form synaptic structures with normal neurons as a signaling conduit within the tumor⁵¹⁻⁵⁵. Specifically, this was found to occur via tumor microtubes displaying AMPA receptors (AMPAR), a glutamate receptor subtype^{54,55}. AMPARs are tetrameric, and there are four subunit proteins involved, Glut1-4, encoded by the genes GRIA1-4 respectively^{56,57}. Interestingly, we found the GRIA genes to be differentially expressed between the different tumor clones in our sample (FIGS. 5B-5E). The major clone, clone 3, expresses GRIA2-4 and does not express GRIA1; clone 1, however, is the only tumor subpopulation that expresses GRIA1; all three other GRIA-family genes are expressed at much lower levels. GRIA1-encoded GluA1 subunit often forms GluA1 homomer AMPARs, which are calcium-permeable and broadly found in synapses in early development⁵⁷. Calcium-permeable AMPARs are a key signaling molecule in the tumor-neuron synapse, and the maintenance of long-term potentiation is also known to be modulated through post-translational modification of GluA1, making GluA1 critical for neural plasticity in the brain^{56, 57}. Clone 1 expression generally resembles astrocytes, including expression of the astrocytic marker APOE (FIG. 12B), but normal astrocytes do not express GRIA1 (FIGS. 5B-5E; FIG. 12B), suggesting a unique, possibly multi-faceted role for clone 1 in cell-cell communication in the tumor microenvironment^{7,54,55}. Next, we also performed ligand-receptor analysis for the different subpopulations and found TGF-beta signaling transcripts to be strongly and specifically expressed in normal astrocytes, clone 1 cells, and tumor-associated macrophages (TAM), with clone 1 cells expressing the ligand and predominantly the TAMs expressing the receptor (FIG. 5F-5I; FIG. 12C-12G). This suggests that clone 1 cells could play a comparable role as normal astrocytes in TGF-beta signaling within the tumor, specifically in modulating immune cell activity.

### EXEMPLIFIED EMBODIMENTS

The invention may be better understood by reference to certain illustrative examples, including but not limited to the following:

Embodiment 1. A method for the amplification of at least one RNA sequence and at least one DNA sequence from a sample, comprising:
a) providing a sample containing at least one RNA sequence and at least one DNA sequence;
b) optionally, purifying the RNA sequence and the DNA sequence from the sample;
c) fragmenting the DNA by contacting the DNA sequence with a transposase loaded with a first DNA oligonucleotide adapter, wherein the transposase fragments the DNA sequence and ligates the first DNA oligonucleotide adapter to the DNA sequence to produce a labelled and fragmented DNA sequence (fDNA), wherein each DNA oligonucleotide adapter comprises a DNA-specific barcode, a shared amplification primer sequence and a unique molecular identifier (UMI);
d) annealing a second DNA oligonucleotide adapter to the RNA sequence, wherein the second DNA oligonucleotide adapter comprises an RNA-specific barcode, the shared amplification primer sequence, an annealing sequence, and a unique molecular identifier (UMI);
e) adding reverse transcriptase to the RNA sequence annealed to the DNA oligonucleotide adapter to synthesize a cDNA sequence;
f) adding a poly C tail to the cDNA sequence;
g) annealing the third DNA oligonucleotide adapter to the polyC-tailed cDNA resulting from step f), wherein the third DNA oligonucleotide adapter comprises a 5′ polyG sequence, an RNA-specific barcode, the shared amplification primer sequence, an annealing sequence, and a unique molecular identifier;
h) synthesizing a DNA sequence complementary to the cDNA sequence of step g) to produce double stranded cDNA; and
i) amplifying the double stranded cDNA sequence and the fDNA sequence simultaneously using the shared primer sequence.

Embodiment 2. The method of embodiment 1, wherein the sample comprises a single cell and/or a nucleus.

Embodiment 3. The method of embodiment 2, wherein the single cell is a bacterial cell, an archaeal cell, or a eukaryotic cell.

Embodiment 4. The method of embodiment 2, wherein step b) further comprises lysing the cell to isolate the RNA sequence and the DNA sequence from the cell.

Embodiment 5. The method of embodiment 1, wherein step c) further comprises providing a plurality of adapters that anneal to the RNA sequence and/or the DNA sequence in the sample.

Embodiment 6. The method of embodiment 5, wherein the plurality of adapters is between about 2 and about 100, about 2 to about 5, or about 4.

Embodiment 7. The method of embodiment 5, wherein step d) further comprises providing at least 2 or at least 3 adapters that anneal to two or more RNA sequences in the sample.

Embodiment 8. The method of embodiment 1, wherein the first, second, or third DNA oligonucleotide adapters further comprise a mosaic sequence and a Seq-1 primer sequence.

Embodiment 9. The method of embodiment 1, wherein the transposase is a Tn5 transposase.

Embodiment 10. The method of embodiment 1, wherein steps a)-i) are carried out in one container.

Embodiment 11. The method of embodiment 1, further comprising:
j) fragmenting the cDNA and fDNA by contacting the cDNA and fDNA sequences with a transposase loaded with a fourth DNA oligonucleotide adapter, wherein the transposase fragments the cDNA and fDNA sequences and ligates the fourth DNA oligonucleotide adapter to the cDNA and fDNA sequences to produce a DNA library, wherein the fourth DNA oligonucleotide adapter comprises a mosaic and DNA annealing sequence, wherein the DNA annealing sequence is complementary to a sequencing primer.

Embodiment 12. The method of embodiment 11, further comprising:
k) sequencing the amplified cDNA sequence and fDNA, wherein the DNA-specific barcode is used to identify DNA sequences and the RNA-specific barcode is used to identify RNA sequences in the resulting sequenced data.

Embodiment 13. A set of oligonucleotide adapters, wherein each adapter comprises an amplification primer sequence, a DNA-specific or RNA-specific barcode, a unique molecular identifier sequence, and an annealing sequence, wherein one oligonucleotide adapter has RNA-specific barcode and the other oligonucleotide adapter has an RNA-specific barcode.

Embodiment 14. The set of oligonucleotide adapters of embodiment 13, wherein the adapters further comprise a mosaic and a Seq-1 primer.

Embodiment 15. An oligonucleotide adapter, wherein the adapter comprises an amplification primer sequence, a DNA-specific or RNA-specific barcode, a unique molecular identifier sequence, and an annealing sequence.

Embodiment 16. The oligonucleotide adapter of embodiment 15, wherein the adapter further comprises a mosaic and/or a Seq-1 primer.

Embodiment 17. The oligonucleotide of embodiment 16, wherein the adapter comprises a nucleotide sequence of SEQ ID NO: 1, SEQ ID NO: 4, SEQ ID NO: 5, or SEQ ID NO: 6 or a nucleotide sequence having at least 95% identity to the nucleotide sequence of SEQ ID NO: 1, SEQ ID NO: 4, SEQ ID NO: 5, or SEQ ID NO: 6.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1 Wu, A. R., Wang, J., Streets, A. M. & Huang, Y. Single-cell transcriptional analysis. Annual Review of Analytical Chemistry 10, 439-462 (2017).
2. Gawad, C., Koh, W. & Quake, S. R. Single-cell genome sequencing: Current state of the science. Nature Reviews Genetics 17, 175-188 (2016).
3. Nam, A. S., Chaligne, R. & Landau, D. A. Integrating genetic and non-genetic determinants of cancer evolution by single-cell multi-omics. Nature Reviews Genetics 22, 3-18 (2021).
4. Birnbaum, K. D. Power in numbers: Single-cell RNA-seq strategies to dissect complex tissues. Annual Review of Genetics 52, 203-221 (2018).
5. Villani, A. C. et al. Single-cell RNA-seq reveals new types of human blood dendritic cells, monocytes, and progenitors. Science 356, (2017).
6. Treutlein, B. et al. Reconstructing lineage hierarchies of the distal lung epithelium using single-cell RNA-seq. Nature 509, 371-375 (2014). 7 Venteicher, A. S. et al. Decoupling genetics, lineages, and microenvironment in IDH-mutant gliomas by single-cell RNA-seq. Science 355, (2017).
8. McGranahan, N. & Swanton, C. Clonal Heterogeneity and Tumor Evolution: Past, Present, and the Future. Cell vol. 168 613-628 (2017).
9. Prager, B. C., Xie, Q., Bao, S. & Rich, J. N. Cancer Stem Cells: The Architects of the Tumor Ecosystem. Cell Stem Cell vol. 24 41-53 (2019).
10. Kreso, A. & Dick, J. E. Evolution of the cancer stem cell model. Cell Stem Cell vol. 14 275-291 (2014).
11. Shaffer, S. M. et al. Rare cell variability and drug-induced reprogramming as a mode of cancer drug resistance. Nature 546, 431-435 (2017).
12. Neftel, C. et al. An Integrative Model of Cellular States, Plasticity, and Genetics for Glioblastoma. Cell 178, 835-849.e21 (2019).
13. Wang, L. et al. The phenotypes of proliferating glioblastoma cells reside on a single axis of variation. Cancer Discovery 9, 1708-1719 (2019).
14. Weng, Q. et al. Single-Cell Transcriptomics Uncovers Glial Progenitor Diversity and Cell Fate Determinants during Development and Gliomagenesis. Cell Stem Cell 24, 707-723.e8 (2019).
15. Müller, S. et al. Single-cell profiling of human gliomas reveals macrophage ontogeny as a basis for regional differences in macrophage activation in the tumor microenvironment. Genome Biology 18, (2017).
16. Pombo Antunes, A. R. et al. Single-cell profiling of myeloid cells in glioblastoma across species and disease stage reveals macrophage competition and specialization. Nature Neuroscience 24, 595-610 (2021).
17. Hara, T. et al. Interactions between cancer cells and immune cells drive transitions to mesenchymal-like states in glioblastoma. Cancer Cell 39, 779-792.e11 (2021).
18. Zhang, L. et al. Single-Cell Analyses Inform Mechanisms of Myeloid-Targeted Therapies in Colon Cancer. Cell 181, 442-459.e29 (2020).
19. Cheng, S. et al. A pan-cancer single-cell transcriptional atlas of tumor infiltrating myeloid cells. Cell 184, 792-809.e23 (2021).
20. Minussi, D. C. et al. Breast tumours maintain a reservoir of subclonal diversity during expansion. Nature 592, 302-308 (2021).
21. Gao, R. et al. Punctuated copy number evolution and clonal stasis in triple-negative breast cancer. Nature Genetics 48, 1119-1130 (2016).
22. Macaulay, I. C. et al. G&T-seq: Parallel sequencing of single-cell genomes and transcriptomes. Nature Methods 12, 519-522 (2015).
23. Dey, S. S., Kester, L., Spanjaard, B., Bienko, M. & van Oudenaarden, A. Integrated genome and transcriptome sequencing of the same cell. Nature Biotechnology 33, 285-289 (2015).
24. Hou, Y. et al. Single-cell triple omics sequencing reveals genetic, epigenetic, and transcriptomic heterogeneity in hepatocellular carcinomas. Cell Research 26, 304-319 (2016).
25. Bian, S. et al. Single-cell multiomics sequencing and analyses of human colorectal cancer. See worldwide website: science.org/doi/10.1126/science.aao3791
26. Zachariadis, V., Cheng, H., Andrews, N. & Enge, M. A Highly Scalable Method for Joint Whole-Genome Sequencing and Gene-Expression Profiling of Single Cells. Molecular Cell 80, 541-553.e5 (2020).
27. Kivioja, T. et al. Counting absolute numbers of molecules using unique molecular identifiers. Nature Methods 9, 72-74 (2012).
28. Klein, A. M. et al. Droplet barcoding for single-cell transcriptomics applied to embryonic stem cells. Cell 161, 1187-1201 (2015).
29. Macosko, E. Z. et al. Highly parallel genome-wide expression profiling of individual cells using nanoliter droplets. Cell 161, 1202-1214 (2015).
30. Ziegenhain, C. et al. Comparative Analysis of Single-Cell RNA Sequencing Methods. Molecular Cell 65, 631-643.e4 (2017).
31. Reznikoff, W. S. Transposon Tn5. Annual Review of Genetics vol. 42 269-286 (2008).
32. Picelli, S. et al. Tn5 transposase and tagmentation procedures for massively scaled sequencing projects. Genome Research 24, 2033-2040 (2014).
33. Hennig, B. P. et al. Large-scale low-cost NGS library preparation using a robust Tn5 purification and tagmentation protocol. G3: Genes, Genomes, Genetics 8, 79-89 (2018).
34. Sheng, K., Cao, W., Niu, Y., Deng, Q. & Zong, C. Effective detection of variation in single-cell transcriptomes using MATQ-seq. Nature Methods 14, 267-270 (2017).
35. Tang, F. et al. mRNA-Seq whole-transcriptome analysis of a single cell. Nature Methods 6, 377-382 (2009).
36. Picelli, S. et al. Smart-seq2 for sensitive full-length transcriptome profiling in single cells. Nature methods 10, 1096-8 (2013).
37. Picelli, S. et al. Full-length RNA-seq from single cells using Smart-seq2. Nature protocols 9, 171-81 (2014).
38. Fan, X. et al. Single-cell RNA-seq transcriptome analysis of linear and circular RNAs in mouse preimplantation embryos. Genome Biology 16, (2015).
39. Garvin, T. et al. Interactive analysis and assessment of single-cell copy-number variations. Nature Methods vol. 12 1058-1060 (2015).
40. Sun, J. C. & Lanier, L. L. NK cell development, homeostasis and function: Parallels with CD8+ T cells. Nature Reviews Immunology vol. 11 645-657 (2011).
41. Farber, D. L., Yudanin, N. A. & Restifo, N. P. Human memory T cells: Generation, compartmentalization and homeostasis. Nature Reviews Immunology vol. 14 24-35 (2014).
42. Pizzolato, G. et al. Single-cell RNA sequencing unveils the shared and the distinct cytotoxic hallmarks of human TCRVδ1 and TCRVδ2 γδ T lymphocytes. Proceedings of the National Academy of Sciences of the United States of America 116, 11906-11915 (2019).
43. Uhlen, M. et al. A genome-wide transcriptomic analysis of protein-coding genes in human blood cells. Science 366, (2019).
44. Lin, W. et al. Establishment and characterization of new tumor xenografts and cancer cell lines from EBV-positive nasopharyngeal carcinoma. Nature Communications 9, (2018).
45. Ben-David, U. et al. Genetic and transcriptional evolution alters cancer cell line drug response. Nature 560, 325-330 (2018).
46. Ceccarelli, M. et al. Molecular Profiling Reveals Biologically Discrete Subsets and Pathways of Progression in Diffuse Glioma. Cell 164, 550-563 (2016).
47. Hu, H. et al. Mutational Landscape of Secondary Glioblastoma Guides MET-Targeted Trial in Brain Tumor. Cell 175, 1665-1678.e18 (2018).
48. Habib, N. et al. Massively parallel single-nucleus RNA-seq with DroNc-seq. Nature Methods 14, 955-958 (2017).
49. Sanchez-Vega, F. et al. Oncogenic Signaling Pathways in The Cancer Genome Atlas. Cell 173, 321-337.e10 (2018).
50. Wang, J. et al. Clonal evolution of glioblastoma under therapy. Nature Genetics 48, 768-776 (2016).
51. Venkataramani, V., Tanev, D. I., Kuner, T., Wick, W. & Winkler, F. Synaptic input to brain tumors: clinical implications. Neuro-oncology 23, 23-33 (2021).
52. Jung, E. et al. Emerging intersections between neuroscience and glioma biology. Nature Neuroscience vol. 22 1951-1960 (2019).
53. Venkatesh, H. S. et al. Electrical and synaptic integration of glioma into neural circuits. Nature 573, 539-545 (2019).
54. Venkataramani, V. et al. Glutamatergic synaptic input to glioma cells drives brain tumour progression. Nature 573, 532-538 (2019).
55. Osswald, M. et al. Brain tumour cells interconnect to a functional and resistant network. Nature 528, 93-98 (2015).
56. Henley, J. M. & Wilkinson, K. A. Synaptic AMPA receptor composition in development, plasticity and disease. Nature Reviews Neuroscience vol. 17 337-350 (2016).
57. Diering, G. H. & Huganir, R. L. The AMPA Receptor Code of Synaptic Plasticity. Neuron vol. 100 314-329 (2018).
58. Tirosh, I. et al. Single-cell RNA-seq supports a developmental hierarchy in human oligodendroglioma. Nature 539, 309-313 (2016).
59. Neftel, C. et al. An Integrative Model of Cellular States, Plasticity, and Genetics for Glioblastoma. Cell 178, 835-849.e21 (2019).
60. Buenrostro, J. D. et al. Single-cell chromatin accessibility reveals principles of regulatory variation. Nature 523, 486-490 (2015).
61. Cao, J. et al. SINGLE-CELL GENOMICS Joint profiling of chromatin accessibility and gene expression in thousands of single cells. Science vol. 361 (2018).
62. Lareau, C. A. et al. Droplet-based combinatorial indexing for massive-scale single-cell chromatin accessibility. Nature Biotechnology 37, 916-924 (2019).
63. Stoeckius, M. et al. Simultaneous epitope and transcriptome measurement in single cells. Nature Methods 14, 865-868 (2017).
64. Corces, M. R. et al. An improved ATAC-seq protocol reduces background and enables interrogation of frozen tissues. Nature Methods 14, 959-962 (2017).
65. Matson, K. J. E. et al. Isolation of adult spinal cord nuclei for massively parallel single-nucleus RNA sequencing. Journal of Visualized Experiments 2018, (2018).
66. Gu, W. et al. Depletion of Abundant Sequences by Hybridization (DASH): Using Cas9 to remove unwanted high-abundance species in sequencing libraries and molecular counting applications. Genome Biology 17, (2016).
67. Loi, D. S. C., Yu, L. & Wu, A. R. Effective ribosomal RNA depletion for single-cell total RNA-seq by scDASH. PeerJ 9, e10717 (2021).
68. Chen, S., Zhou, Y., Chen, Y. & Gu, J. Fastp: An ultra-fast all-in-one FASTQ preprocessor. in Bioinformatics vol. 34 (2018).
69. Shen, W., Le, S., Li, Y. & Hu, F. SeqKit: A cross-platform and ultrafast toolkit for FASTA/Q file manipulation. PLoS ONE 11, (2016).
70. Li, H. seqtk Toolkit for processing sequences in FASTA/Q formats. GitHub 767, (2012).
71. Bushnell, B., Rood, J. & Singer, E. BBTools Software Package. PLOS ONE vol. 12 e0185056 https://sourceforge.net/projects/bbmap/(2017).
72. Li, H. & Durbin, R. Fast and accurate short read alignment with Burrows-Wheeler transform. Bioinformatics 25, (2009).
73. Li, H. et al. The Sequence Alignment/Map format and SAMtools. Bioinformatics 25, (2009).
74. Smith, T., Heger, A. & Sudbery, I. UMI-tools: Modeling sequencing errors in Unique Molecular Identifiers to improve quantification accuracy. Genome Research 27, (2017).
75. Quinlan, A. R. & Hall, I. M. BEDTools: A flexible suite of utilities for comparing genomic features. Bioinformatics 26, (2010).
76. Garvin, T. et al. Interactive analysis and assessment of single-cell copy-number variations. Nature Methods vol. 12 1058-1060 (2015).
77. Zaccaria, S. & Raphael, B. J. Characterizing allele- and haplotype-specific copy numbers in single cells with CHISEL. Nature Biotechnology 39, 207-214 (2021).
78. Das, S. et al. Next-generation genotype imputation service and methods. Nature Genetics 48, (2016).
79. Gao, R. et al. Punctuated copy number evolution and clonal stasis in triple-negative breast cancer. Nature Genetics 48, 1119-1130 (2016).
80. Navin, N. et al. Tumour evolution inferred by single-cell sequencing. Nature 472, 90-95 (2011).
81. Minussi, D. C. et al. Breast tumours maintain a reservoir of subclonal diversity during expansion. Nature 592, 302-308 (2021).
82. Nilsen, G. et al. Copynumber: Efficient algorithms for single- and multi-track copy number segmentation. BMC Genomics 13, (2012).
83. Dobin, A. et al. STAR: Ultrafast universal RNA-seq aligner. Bioinformatics 29, (2013).
84. Bray, N. L., Pimentel, H., Melsted, P. & Pachter, L. Near-optimal probabilistic RNA-seq quantification. Nature Biotechnology 34, (2016).
85. Patro, R., Duggal, G., Love, M. I., Irizarry, R. A. & Kingsford, C. Salmon provides fast and bias-aware quantification of transcript expression. Nature Methods 14, (2017).
86. Melsted, P. et al. Modular, efficient and constant-memory single-cell RNA-seq preprocessing. Nature Biotechnology 39, (2021).
87. Butler, A., Hoffman, P., Smibert, P., Papalexi, E. & Satija, R. Integrating single-cell transcriptomic data across different conditions, technologies, and species. Nature Biotechnology 36, (2018).
88. Stuart, T. et al. Comprehensive Integration of Single-Cell Data. Cell 177, 1888-1902.e21 (2019).
89. Finak, G. et al. MAST: A flexible statistical framework for assessing transcriptional changes and characterizing heterogeneity in single-cell RNA sequencing data. Genome Biology 16, (2015).
90. Neftel, C. et al. An Integrative Model of Cellular States, Plasticity, and Genetics for Glioblastoma. Cell 178, 835-849.e21 (2019).
91. Gao, R. et al. Delineating copy number and clonal substructure in human tumors from single-cell transcriptomes. Nature Biotechnology 39, (2021).
92. Jin, S. et al. Inference and analysis of cell-cell communication using Cell Chat. Nature Communications 2021 12:1 12, 1-20 (2021).
93. Gómez-Rubio, V. ggplot2—Elegant Graphics for Data Analysis (2nd Edition). Journal of Statistical Software 77, (2017).
94. Kassambara, A. ggpubr: “ggplot2” Based Publication Ready Plots. R package version 0.4.0 (2020).
95. Gu, Z., Eils, R. & Schlesner, M. Complex heatmaps reveal patterns and correlations in multidimensional genomic data. Bioinformatics 32, (2016).
96. Inkscape.org. Draw Freely|Inkscape. inkscape.org (2020).

1. A method for the amplification of at least one RNA sequence and at least one DNA sequence from a sample, comprising:
a) providing a sample containing at least one RNA sequence and at least one DNA sequence;
b) optionally, purifying the RNA sequence and the DNA sequence from the sample;
c) fragmenting the DNA by contacting the DNA sequence with a transposase loaded with a first DNA oligonucleotide adapter, wherein the transposase fragments the DNA sequence and ligates the first DNA oligonucleotide adapter to the DNA sequence to produce a labelled and fragmented DNA sequence (fDNA), wherein each DNA oligonucleotide adapter comprises a DNA-specific barcode, a shared amplification primer sequence, and a unique molecular identifier (UMI);
d) annealing a second DNA oligonucleotide adapter to the RNA sequence, wherein the second DNA oligonucleotide adapter comprises an RNA-specific barcode, the shared amplification primer sequence, an annealing sequence, and a unique molecular identifier (UMI);
e) adding reverse transcriptase to the RNA sequence annealed to the DNA oligonucleotide adapter to synthesize a cDNA sequence;
f) adding a poly C tail to the cDNA sequence;
g) annealing the third DNA oligonucleotide adapter to the polyC-tailed cDNA resulting from step f), wherein the third DNA oligonucleotide adapter comprises a 5′ polyG sequence, an RNA-specific barcode, the shared amplification primer sequence, an annealing sequence, and a unique molecular identifier;
h) synthesizing a DNA sequence complementary to the cDNA sequence of step g) to produce double stranded cDNA; and
i) amplifying the double stranded cDNA sequence and the fDNA sequence simultaneously using the shared primer sequence.

2. The method of claim 1, wherein the sample comprises a single cell and/or a nucleus.

3. The method of claim 2, wherein the single cell is a bacterial cell, an archaeal cell, or a eukaryotic cell.

4. The method of claim 2, wherein step b) further comprises lysing the cell to isolate the RNA sequence and the DNA sequence from the cell.

5. The method of claim 1, wherein step c) further comprises providing a plurality of adapters that anneal to the RNA sequence and/or the DNA sequence in the sample.

6. The method of claim 5, wherein the plurality of adapters is between about 2 and about 100, about 2 to about 5, or about 4.

7. The method of claim 5, wherein step d) further comprises providing at least 2 or at least 3 adapters that anneal to two or more RNA sequences in the sample.

8. The method of claim 1, wherein the first, second, or third DNA oligonucleotide adapters further comprise a mosaic sequence and a Seq-1 primer sequence.

9. The method of claim 1, wherein the transposase is a Tn5 transposase.

10. The method of claim 1, wherein steps a)-i) are carried out in one container.

11. The method of claim 1, further comprising:
j) fragmenting the cDNA and fDNA by contacting the cDNA and fDNA sequences with a transposase loaded with a fourth DNA oligonucleotide adapter, wherein the transposase fragments the cDNA and fDNA sequences and ligates the fourth DNA oligonucleotide adapter to the cDNA and fDNA sequences to produce a DNA library, wherein the fourth DNA oligonucleotide adapter comprises a mosaic and DNA annealing sequence, wherein the DNA annealing sequence is complementary to a sequencing primer.

12. The method of claim 11, further comprising:
k) sequencing the amplified cDNA sequence and fDNA, wherein the DNA-specific barcode is used to identify DNA sequences and the RNA-specific barcode is used to identify RNA sequences in the resulting sequenced data.

13. A set of oligonucleotide adapters, wherein each adapter comprises an amplification primer sequence, a DNA-specific or RNA-specific barcode, a unique molecular identifier sequence, and an annealing sequence, wherein one oligonucleotide adapter has DNA-specific barcode and the other oligonucleotide adapter has an RNA-specific barcode.

14. The set of oligonucleotide adapters of claim 13, wherein the adapters further comprise a mosaic and a Seq-1 primer.

15. An oligonucleotide adapter, wherein the adapter comprises an amplification primer sequence, a DNA-specific or RNA-specific barcode, a unique molecular identifier sequence, and an annealing sequence.

16. The oligonucleotide adapter of claim 15, wherein the adapter further comprises a mosaic and/or a Seq-1 primer.

17. The oligonucleotide of claim 16, wherein the adapter comprises a nucleotide sequence of SEQ ID NO: 1, SEQ ID NO: 4, SEQ ID NO: 5, or SEQ ID NO: 6 or a nucleotide sequence having at least 95% identity to the nucleotide sequence of SEQ ID NO: 1, SEQ ID NO: 4, SEQ ID NO: 5, or SEQ ID NO: 6.
